(19) United States
(12) Patent Application Publication
(10) Pub. No.: US 2024/0269111 A1
(43) Pub. Date: Aug. 15, 2024

(54) METHODS AND COMPOSITIONS FOR TREATING OCULAR NEOVASCULAR DISEASE

(71) Applicant: METANOIA BIO INC., Beverly Hills, CA (US)

(72) Inventors: Margaretha ROUX, Beverly Hills, CA (US); Slavica TUDZAROVA-TRAJKOVSKA, Beverly Hills, CA (US); Abraham VAN WYK, Beverly Hills, CA (US)

(21) Appl. No.: 18/561,103

(22) PCT Filed: May 16, 2022
(86) PCT No.: PCT/US2022/029387

§ 371 Date: Nov. 15, 2023

Related U.S. Application Data
(60) Provisional application No. 63/189,204, filed on May 16, 2021.

(51) Int. Cl. A61K 31/404; A61K 31/196; A61K 31/245; A61K 31/352; A61K 31/381; A61K 31/403; A61K 31/41; A61K 31/4155; A61K 31/422; A61K 31/427; A61K 31/444; A61K 31/47; A61K 31/4709; A61K 31/498; A61K 31/501; A61K 31/506; A61K 31/519; A61K 45/06

(52) U.S. Cl. CPC A61K 31/404; A61K 31/196; A61K 31/245; A61K 31/352; A61K 31/381; A61K 31/403; A61K 31/41; A61K 31/4155; A61K 31/422; A61K 31/427; A61K 31/444; A61K 31/47; A61K 31/4709; A61K 31/498; A61K 31/501; A61K 31/506; A61K 31/519; A61K 45/06

(57) ABSTRACT

The disclosure provides compositions and methods for treating ocular neovascular diseases and conditions with HIF1-α Pathway Inhibitors and PFKFB3 Inhibitors. Exemplary ocular neovascular diseases and conditions treated using the provided compositions and methods include diabetic retinopathy, diabetic macular edema, age-related macular degeneration, and choroidal neovascular membranes.

### BACKGROUND

Retinal and choroidal vascular diseases constitute the most common causes of moderate and severe vision loss in developed countries. They can be divided into retinal vascular diseases, in which there is leakage and/or neovascularization from retinal vessels, and subretinal neovascularization, in which new vessels grow into the normally avascular outer retina and subretinal space. The first category of diseases includes diabetic retinopathy, diabetic macular edema retinal vein occlusions, and retinopathy of prematurity and the second category includes neovascular age-related macular degeneration (AMD), ocular histoplasmosis, pathologic myopia, and other related diseases.

Diabetic retinopathy (DR) is the leading cause of blindness in adults of developed countries. DR is a severe complication of diabetes mellitus that is the most common diabetic eye disease, and often leads to diminution of vision or blindness. According to statistics, 50% of diabetics will have DR in about 10 years of the disease course, and up to 80% of diabetics will have DR in 15 years or more of disease course. The more severe the condition of diabetes mellitus is and the older the patient is, the higher the incidence of DR.

Both microvasculopathy and neurodegeneration are implicated in mechanisms of DR development, with neuronal impairment preceding microvascular abnormalities, which is underappreciated in the clinic. Traditionally, vasculopathy has been considered the primary pathophysiologic mechanism responsible for diabetic retinopathy (DR). However, in recent years, the role of diabetic retinal neurodegeneration (DRN) is increasingly evident and quite possibly supersedes that of vasculopathy as the primary pathogenic event of the disease.

Diabetic macular edema (DME) is a complication of DR that affects up to 10% of people with diabetes and is the most frequent cause of sight loss in people with DR.

The mechanisms of diabetic retinopathy and diabetic macular edema and therapeutic strategies for treating them are the subject of extensive efforts. In a clinical setting, for example, laser photocoagulation, corticosteroids and antiangiogenic therapy using drugs that bind to an sequester vascular endothelial growth (e.g., bevacizumab and ranibizumab), represent state-of-the-art therapeutic strategies for inducing angiogenic regression and reduction of DR and macular edema.

Neurodegeneration is a common pathway of assorted processes, including activation of inflammatory pathways, reduction of neuroprotective factors, DNA damage, and apoptosis. Oxidative stress and formation of advanced glycation end products amplify these processes and are elevated in the setting of hyperglycemia, hyperlipidemia, and glucose variability.

The impairment of the neurosensory retina in diabetes is governed by various mechanisms, which may be classified as inflammatory, metabolic, and genetic/epigenetic.

Age-related macular degeneration (AMD) is a degenerative ocular disease affecting macula in the retina that is a notable cause of vision loss in the U.S. population among persons 65 years and older. Neovascular or exudative or wet AMD (nAMD, wAMD, or nwAMD) is an advanced form of AMD. The hallmark of wAMD is choroidal neovascularization (CNVM), which is the infiltration of abnormal blood vessels in the retina from the underlying choroid layer, resulting in retinal cell damage and central blindness. CNVM is also prevalent in ocular disorders such as histoplasmosis, eye trauma and myopic macular degeneration These abnormal angiogenic processes are typically modulated by photodynamic therapy, thermal laser treatment and treatment with growth factors, in particular, molecules that bind to and sequester VEGF, such as ranibizumab (e.g., LUCENTIS®) and aflibercept (e.g., EYLEA®).

Most current therapeutic strategies for ocular neovascular disorders (e.g., diabetic retinopathy, diabetic macular edema, age-related wet macular degeneration, and choroidal neovascular membranes) such as anti-vascular endothelial growth factor (anti-VEGF)-antibodies, aim at treating the advanced stages (diabetic macular oedema and proliferative diabetic retinopathy) and fail to target the neuronal deterioration. The prevention and treatment of the neurodegenerative component of DR is thus overlooked, though the insidious loss of neurons is irreversible. These strategies do not address preventing or treating the early stages of the early stages of these disorders. Moreover, many patients are unresponsive to the current therapeutic approaches for treating ocular neovascular disorders and the state of the art anti-angiogenic and photocoagulation therapies are accompanied by significant side-effects. In addition, the resistance to anti-VEGF antibodies has been reported in connection with FGF substituting for the role of VEGF in promoting angiogenesis.

Accordingly, new modalities for treating both the vascular and neuronal impairment in ocular neovascular disease such as diabetic retinopathy, age-related wet macular degeneration, and choroidal neovascular membranes are needed. The compositions and methods provided herein address these needs and provide other related advantages.

### BRIEF SUMMARY

The disclosure provides compositions and methods for treating ocular neovascular diseases and conditions with HIF1-α Pathway Inhibitors and PFKFB3 Inhibitors. Exemplary ocular neovascular diseases and conditions treated using the provided compositions and methods include diabetic retinopathy, diabetic macular edema, age-related macular degeneration, and choroidal neovascular membranes.

The inventors have determined that the Hypoxia-inducible factor 1α (HIF1-α)-6-phosphofructo-2-kinase-fructose-2,6-bisphosphatase 3 (PFKFB3) pathway plays a central role in pathologic angiogenesis and neurodegeneration and have surprisingly found that the combination of an a HIF1-α Pathway Inhibitor and an PFKFB3 inhibitor is able to mitigate and possibly even reverse the damage caused by these pathologies. Pathologic angiogenesis and neurodegeneration are two key aspects in the diabetic retinopathy complication and the HIF1-α-PFKFB3 signalling pathway is unique as being a pervasive pathological component across multiple cell types in the retina in the early as well as late stages of DR and other ocular neovascular disorder.

In some embodiments, the disclosure provides:
[1] a method of treating an ocular neovascular disease or condition in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor;
   wherein the PFKFB3 inhibitor does not inhibit PI3K/AKT/mTOR pathway or HIF1-α;
[2] the method of [1], wherein the subject is administered an effective amount of the HIF1-α Pathway Inhibitor and the PFKFB3 inhibitor;
[3] the method of [1], wherein the subject is administered an effective amount of the HIF1-α Pathway Inhibitor and wherein the subject has previously been administered the PFKFB3 Inhibitor;
[4] the method of [1], wherein the subject is administered an effective amount of the PFKFB3 Inhibitor and wherein the subject has previously been administered the HIF1-α Pathway Inhibitor;
[5] the method of any one of [1]-[4], wherein the subject has or is at risk of having the ocular neovascular disease or condition;
[6] the method of any one of [1]-[4], wherein the subject has or has been diagnosed as having the ocular neovascular disease or condition;
[7] the method of any one of [1]-[5], wherein the method of any one of 1(a)-1(c) is administered as a prophylactic treatment for the ocular neovascular disease or condition;
[8] the method of any one of [1]-[7], wherein the ocular neovascular disease or condition is diabetic retinopathy (DR);
[9] the method of any one of [1]-[8], wherein the ocular neovascular disease or condition is diabetic macular edema (DME);
[10] the method of any one of [1]-[7], wherein the ocular neovascular disease or condition is age-related macular degeneration (AMD), such as wet AMD (wAMD);
[11] the method of any one of [1]-[7], wherein the ocular neovascular disease or condition is choroidal neovascular membranes;
[12] the method of any one of [1]-[11], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor;
[13] the method of any one of [1]-[12], wherein the administered HIF1-α Pathway Inhibitor is silibinin, PX-478 or YC-1.3, or a salt thereof;
[14] the method of any one of [1]-[13], wherein the administered HIF1-α Pathway Inhibitor is ganetespib (ST-9090), phenethyl isothiocyanate, or BAY-87-2243, or a salt thereof;
[15] the method of any one of [1]-[14], wherein the administered HIF1-α Pathway Inhibitor is a HIF1-α Inhibitor;
[16] the method of [15], wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor;
[17] the method of [15] or [16], wherein the administered HIF1-α Inhibitor is antisense oligonucleotide EZN-2968 or nanobody AG-1, AG-2, AG-3, AG-4, AG-5, VHH212, or AHPC;
[18] the method of any one of [1]-[17], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[19] the method of any one of [1]-[18[, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[20] the method of any one of [1]-[19], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[21] the method of any one of [1]-[20], wherein the HIF1-α Pathway Inhibitor and the PFKFB3 inhibitor are co-administered to the subject;
[22] the method of any one of [1]-[21], wherein the administration of the HIF1-α Pathway Inhibitor or the PFKFB3 inhibitor is an ocular administration;
[23] the method of any one of [1]-[22], wherein the administration of the HIF1-α Pathway Inhibitor and the PFKFB3 inhibitor is an ocular administration;
[24] the method of [22] or [23], wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration;
[25] the method of any one of [22]-[24], wherein the administration of the HIF1-α Pathway Inhibitor or the PFKFB3 inhibitor is intravitreal administration;
[26] the method of any one of [22]-[25], wherein the administration of the HIF1-α Pathway Inhibitor and the PFKFB3 inhibitor is intravitreal administration;
[27] the method of any one of [1]-[26], wherein the HIF1-α Pathway Inhibitor and the PFKFB3 inhibitor are administered before the onset of one or more symptoms of the ocular neovascular disease or condition;
[28] the method of any one of [1]-[26], wherein the HIF1-α Pathway Inhibitor and the PFKFB3 inhibitor are administered after the onset of one or more symptoms of the ocular neovascular disease or condition;
[29] the method of any one of [1]-[26], wherein the HIF1-α Pathway Inhibitor and the PFKFB3 inhibitor are administered during a non-proliferative stage of the ocular neovascular disease or condition;
[30] the method of any one of [1]-[26], wherein the HIF1-α Pathway Inhibitor and the PFKFB3 inhibitor are administered during a proliferative stage of the ocular neovascular disease or condition;
[31] the method of any one of [1]-[27], wherein treating the ocular neovascular disease or condition comprises delaying the onset of the ocular neovascular disease or condition;
[32] the method of any one of [1]-[31], wherein one or more symptoms of the ocular neovascular disease or condition is reduced in the subject compared to a control subject or compared to the subject prior to treatment with the HIF1-α Pathway Inhibitor and the PFKFB3 inhibitor;
[33] the method of [32], wherein the one or more symptoms of the ocular neovascular disease or condition is selected from: retinal inflammation, acellular capillary formation, retinal neovascularization, retinal endothelial cell death, retinal vascular permeability, retinal ischemia-reperfusion injury, retinal leakage area, choroidal inflammation, choroidal neovascularization, choroidal endothelial cell death, choroidal vascular permeability, choroidal ischemia-reperfusion injury, choroidal leakage area, and occludin disruption and occludin disruption;
[34] the method of [32] or [33], wherein the one or more symptoms of the ocular neovascular disease or condition are reduced by at least 10%, at least 20%, at least 30%, at least 40%, or at least 50% compared to the control subject or compared to the subject prior to treatment with the HIF1-α Pathway Inhibitor and the PFKFB3 inhibitor;
[35] the method of any one of [1]-[34], wherein one or more vision parameters are increased in the subject compared to the vision parameters in a control subject or compared to the vision parameters of the subject prior to treatment with the HIF1-α Pathway Inhibitor and the PFKFB3 inhibitor;
[36] the method of [35], wherein the one or more vision parameters are selected from peripheral vision; night vision; low light vision, color vision; distance vision; close-range vision; vision field clarity, ability to read, absence of flashing lights or spots in the vision filed, non-fluctuating vision, pain, and eye appearance;
[37] the method of any one of [1]-[36], which further comprises administering an anti-VEGF therapeutic agent to the subject;
[38] the method of [37], wherein the anti-VEGF therapeutic agent is bevacizumab, ranibizumab, or aflibercept;
[39] the method of any one of [1]-[38], wherein the subject has received a prior treatment for the ocular neovascular disease or condition;
[40] the method of [39], wherein the subject failed to respond to the prior treatment for the ocular neovascular disease or condition;
[41] the method of [39] or [40], wherein the prior treatment is a therapeutic procedure selected from a vitrectomy and laser surgery, or a therapeutic agent selected from a steroid and an anti-vascular endothelial growth factor (VEGF) therapy;
[42] the method of [41], wherein the prior treatment is an anti-vascular endothelial growth factor (VEGF) therapy;
[43] the method of [42], wherein the anti-VEGF therapy is the administration of bevacizumab, ranibizumab, or aflibercept;
[44] a method of treating diabetic retinopathy (DR) in a subject in need thereof comprising:
   (a) administering an effective amount of a HIF1-α Pathway Inhibitor and a PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor;
   wherein the PFKFB3 inhibitor does not inhibit PI3K/AKT/mTOR pathway or HIF1-α;
[45] the method of [44], wherein the subject is administered an effective amount of the HIF1-α Pathway Inhibitor and the PFKFB3 inhibitor;
[46] the method of [44], wherein the subject is administered an effective amount of the HIF1-α Pathway Inhibitor and wherein the subject has previously been administered the PFKFB3 Inhibitor;
[47] the method of [44], wherein the subject is administered an effective amount of the PFKFB3 Inhibitor and wherein the subject has previously been administered the HIF1-α Pathway Inhibitor;
[48] the method of any one of [44]-[47], wherein the subject has or is at risk of having DR;
[49] the method of any one of [44]-[48], wherein the subject has or has been diagnosed as having DR;
[50] the method of any one of [44]-[48], wherein the method of any one of 44(a)-44(c) is administered as a prophylactic treatment for DR;
[51] the method of any one of [44]-[50], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor;
[52] the method of any one of [44]-[51], wherein the administered HIF1-α Pathway Inhibitor is silibinin, PX-478 or YC-1.3, or a salt thereof;
[53] the method of any one of [44]-[52], wherein the administered HIF1-α Pathway Inhibitor is ganetespib (ST-9090), phenethyl isothiocyanate, or BAY-87-2243, or a salt thereof;
[54] the method of any one of [44]-[53], wherein the administered HIF1-α Pathway Inhibitor is a HIF1-α Inhibitor;
[55] the method of [54], wherein the HIF1-α Inhibitor is HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor;
[56] the method of [54] or [55], wherein the administered HIF1-α Inhibitor is antisense oligonucleotide EZN-2968 or nanobody AG-1, AG-2, AG-3, AG-4, AG-5, VHH212, or AHPC;
[57] the method of any one of [44]-[56], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[58] the method of any one of [44]-[57], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[59] the method of any one of [44]-[57], wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[60] the method of any one of [44]-[59], wherein the HIF1-α Pathway Inhibitor and the PFKFB3 inhibitor are co-administered to the subject;
[61] the method of any one of [44]-[60], wherein the administration of the HIF1-α Pathway Inhibitor or the PFKFB3 inhibitor is an ocular administration;
[62] the method of any one of [44]-[61], wherein the administration of the HIF1-α Pathway Inhibitor and the PFKFB3 inhibitor is an ocular administration;
[63] the method of [61] or [62], wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration;
[64] the method of any one of [61]-[63], wherein the administration of the HIF1-α Pathway Inhibitor or the PFKFB3 inhibitor is intravitreal administration;
[65] the method of any one of [61]-[64], wherein the administration of the HIF1-α Pathway Inhibitor and the PFKFB3 inhibitor is intravitreal administration;
[66] the method of any one of [44]-[65], wherein the HIF1-α Pathway Inhibitor and the PFKFB3 inhibitor are administered before the onset of one or more symptoms of DR;
[67] the method of any one of [44]-[65], wherein the HIF1-α Pathway Inhibitor and the PFKFB3 inhibitor are administered after the onset of one or more symptoms of DR;
[68] the method of any one of [44]-[65], wherein the HIF1-α Pathway Inhibitor and the PFKFB3 inhibitor are administered during a non-proliferative stage of DR;
[69] the method of any one of [44]-[65], wherein the HIF1-α Pathway Inhibitor and the PFKFB3 inhibitor are administered during a proliferative stage of DR;
[70] the method of any one of [44]-[69], wherein treating DR comprises delaying the onset of DR;
[71] the method of any one of [44]-[70], wherein one or more symptoms of DR is reduced in the subject compared to a control subject or compared to the subject prior to treatment with the HIF1-α Pathway Inhibitor and the PFKFB3 inhibitor;
[72] the method of [71], wherein the one or more symptoms of DR is selected from: retinal inflammation, acellular capillary formation, retinal neovascularization, retinal endothelial cell death, retinal vascular permeability, retinal ischemia-reperfusion injury, retinal leakage area, and occludin disruption;
[73] the method of [71] or [72], wherein the one or more symptoms of DR are reduced by at least 10%, at least 20%, at least 30%, at least 40%, or at least 50% compared to the control subject or compared to the subject prior to treatment with the HIF1-α Pathway Inhibitor and the PFKFB3 inhibitor;
[74] the method of any one of [44]-[73], wherein one or more vision parameters are increased in the subject compared to the vision parameters a in a control subject or compared to the vision parameters of the subject prior to treatment with the HIF1-α Pathway Inhibitor and the PFKFB3 inhibitor;
[75] the method of [74], wherein the one or more vision parameters are selected from peripheral vision; night vision; color vision; distance vision; close-range vision; and vision clarity;
[76] the method of any one of [44]-[75], which further comprises administering an anti-VEGF therapeutic agent to the subject;
[77] the method of [76], wherein the anti-VEGF therapeutic agent is bevacizumab, ranibizumab, or aflibercept;
[78] the method of any one of [44]-[77], wherein the subject has received a prior treatment for DR;
[79] the method of [78], wherein the subject failed to respond to the prior treatment for DR;
[80] the method of [78] or [79], wherein the prior treatment is a therapeutic procedure selected from a vitrectomy and laser surgery, or a therapeutic agent selected from a steroid and an anti-vascular endothelial growth factor (VEGF) therapy;
[81] the method of [79] or [80], wherein the prior treatment is an anti-vascular endothelial growth factor (VEGF) therapy;
[82] the method of [81], wherein the anti-VEGF therapy is the administration of bevacizumab, ranibizumab, or aflibercept;
[83] a method of treating Diabetic macular edema (DME) in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor;
   wherein the PFKFB3 inhibitor does not inhibit PI3K/AKT/mTOR pathway or HIF1-α;
[84] the method of [83], wherein the subject is administered an effective amount of the HIF1-α Pathway Inhibitor and the PFKFB3 inhibitor;
[85] the method of [83], wherein the subject is administered an effective amount of the HIF1-α Pathway Inhibitor and wherein the subject has previously been administered the PFKFB3 Inhibitor;
[86] the method of [83], wherein the subject is administered an effective amount of the PFKFB3 Inhibitor and wherein the subject has previously been administered the HIF1-α Pathway Inhibitor;
[87] the method of any one of [83]-[86], wherein the subject has or is at risk of having DME;
[88] the method of any one of [83]-[87], wherein the subject has or has been diagnosed as having DME;
[89] the method of any one of [83]-[87], wherein the method of any one of 83(a)-83(c) is administered as a prophylactic treatment for DME;
[90] the method of any one of [83]-[89], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor;
[91] the method of any one of [83]-[90], wherein the administered HIF1-α Pathway Inhibitor is silibinin, PX-478 or YC-1.3, or a salt thereof;
[92] the method of any one of [83]-[91], wherein the administered HIF1-α Pathway Inhibitor is ganetespib (ST-9090), phenethyl isothiocyanate, or BAY-87-2243, or a salt thereof;
[93] the method of any one of [83]-[92], wherein the administered HIF1-α Pathway Inhibitor is a HIF1-α Inhibitor.
[94] the method of [93], wherein the HIF1-α Inhibitor is HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor;
[95] the method of [93] or [94], wherein the administered HIF1-α Inhibitor is antisense oligonucleotide EZN-2968 or nanobody AG-1, AG-2, AG-3, AG-4, AG-5, VHH212, or AHPC;
[96] the method of any one of [83]-[95], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[97] the method of any one of [83]-[96], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[98] the method of any one of [83]-[96[, wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[99] the method of any one of [83]-[98[, wherein the HIF1-α Pathway Inhibitor and the PFKFB3 inhibitor are co-administered to the subject;
[100] the method of any one of [83]-[99], wherein the administration of the HIF1-α Pathway Inhibitor or the PFKFB3 inhibitor is an ocular administration.
[101] the method of any one of [83]-[100], wherein the administration of the HIF1-α Pathway Inhibitor and the PFKFB3 inhibitor is an ocular administration.
[102] the method of [100[or [101], wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.
[103] the method of any one of [100]-[102], wherein the administration of the HIF1-α Pathway Inhibitor or the PFKFB3 inhibitor is intravitreal administration.
[104] the method of any one of [100]-[103], wherein the administration of the HIF1-α Pathway Inhibitor and the PFKFB3 inhibitor is intravitreal administration.
[105] the method of any one of [83]-[104], wherein the HIF1-α Pathway Inhibitor and the PFKFB3 inhibitor are administered before the onset of one or more symptoms of DME.
[106] the method of any one of [83]-[104], wherein the HIF1-α Pathway Inhibitor and the PFKFB3 inhibitor are administered after the onset of one or more symptoms of DME.
[107] the method of any one of [83]-[104], wherein the HIF1-α Pathway Inhibitor and the PFKFB3 inhibitor are administered during a non-proliferative stage of DME.
[108] the method of any one of [83]-[104] wherein the HIF1-α Pathway Inhibitor and the PFKFB3 inhibitor are administered during a proliferative stage of DME.
[109] the method of any one of [83]-[105], wherein treating DME comprises delaying the onset of DME.
[110] the method of any one of [83]-[109], wherein one or more symptoms of DME is reduced in the subject compared to a control subject or compared to the subject prior to treatment with the HIF1-α Pathway Inhibitor and the PFKFB3 inhibitor.
[111] the method of [110], wherein the one or more symptoms of DME is selected from: distorted vision, retinal inflammation, acellular capillary formation, retinal neovascularization, retinal endothelial cell death, retinal vascular permeability, retinal ischemia-reperfusion injury, retinal leakage area, rupture of the hematorretinal barrier of the retina in a retinal vascular endothelial cell or a retinal pigment epithelial cell, retinal scarring, and occludin disruption.
[112] the method of [110] or [111], wherein the one or more symptoms of DME are reduced by at least 10%, at least 20%, at least 30%, at least 40%, or at least 50% compared to the control subject or compared to the subject prior to treatment with the HIF1-α Pathway Inhibitor and the PFKFB3 inhibitor.
[113] the method of any one of [83]-[112], wherein one or more vision parameters are increased in the subject compared to the vision parameters a in a control subject or compared to the vision parameters of the subject prior to treatment with the HIF1-α Pathway Inhibitor and the PFKFB3 inhibitor.
[114] the method of [113], wherein the one or more vision parameters are selected from visual acuity, peripheral vision; night vision; color vision; distance vision; close-range vision; and vision clarity;
[115] the method of any one of [83]-[114], wherein the subject has diabetes or diabetic retinopathy;
[116] the method of any one of [83]-[115], which further comprises administering an anti-VEGF therapeutic agent to the subject.
[117] the method of [116], wherein the anti-VEGF therapeutic agent is bevacizumab, ranibizumab, or aflibercept;
[118] the method of any one of [83]-[117], wherein the subject has received a prior treatment for DME.
[119] the method of [118], wherein the subject failed to respond to the prior treatment for DME.
[120] the method of [118] or [119], wherein the prior treatment is a therapeutic procedure selected from a vitrectomy and laser surgery, or a therapeutic agent selected from a steroid and an anti-vascular endothelial growth factor (VEGF) therapy.
[121] the method of [118] or [119], wherein the prior treatment is an anti-vascular endothelial growth factor (VEGF) therapy.
[122] the method of [121], wherein the anti-VEGF therapy is the administration of bevacizumab, ranibizumab, or aflibercept.
[123] a method of treating age-related macular degeneration (AMD) in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor;
   wherein the PFKFB3 inhibitor does not inhibit PI3K/AKT/mTOR pathway or HIF1-α.
[124] the method of [123], wherein the subject is administered an effective amount of the HIF1-α Pathway Inhibitor and the PFKFB3 inhibitor;
[125] the method of [123], wherein the subject is administered an effective amount of the HIF1-α Pathway Inhibitor and wherein the subject has previously been administered the PFKFB3 Inhibitor;
[126] the method of [123], wherein the subject is administered an effective amount of the PFKFB3 Inhibitor and wherein the subject has previously been administered the HIF1-α Pathway Inhibitor;
[127] the method of any one of [123]-[126], wherein the subject has or is at risk of having AMD, e.g., wet AMD (wAMD);
[128] the method of any one of [123]-[126], wherein the subject has or has been diagnosed as having AMD e.g., wAMD;
[129] the method of any one of [123]-[127], wherein the method of any one of 123(a)-123(c) is administered as a prophylactic treatment for AMD e.g., wAMD;
[130] the method of any one of [123]-[129], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor;
[131] the method of any one of [123]-[130], wherein the administered HIF1-α Pathway Inhibitor is silibinin, PX-478 or YC-1.3, or a salt thereof;
[132] the method of any one of [123]-[131], wherein the administered HIF1-α Pathway Inhibitor is ganetespib (ST-9090), phenethyl isothiocyanate, or BAY-87-2243, or a salt thereof;
[133] the method of any one of [123]-[132], wherein the administered HIF1-α Pathway Inhibitor is a HIF1-α Inhibitor.
[134] the method of [133], wherein the HIF1-α Inhibitor is HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor;
[135] the method of [133] or [134], wherein the administered HIF1-α Inhibitor is antisense oligonucleotide EZN-2968 or nanobody AG-1, AG-2, AG-3, AG-4, AG-5, VHH212, or AHPC;
[136] the method of any one of [123]-[135], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[137] the method of any one of [123]-[136], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[138] the method of any one of [123]-[136], wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[139] the method of any one of [123]-[138], wherein the HIF1-α Pathway Inhibitor and the PFKFB3 inhibitor are co-administered to the subject;
[140] the method of any one of [123]-[139], wherein the administration of the HIF1-α Pathway Inhibitor or the PFKFB3 inhibitor is an ocular administration.
[141] the method of any one of [123]-[140], wherein the administration of the HIF1-α Pathway Inhibitor and the PFKFB3 inhibitor is an ocular administration.
[142] the method of [140] or [141], wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.
[143] the method of any one of [140]-[142], wherein the administration of the HIF1-α Pathway Inhibitor or the PFKFB3 inhibitor is intravitreal administration.
[144] the method of any one of [140]-[143], wherein the administration of the HIF1-α Pathway Inhibitor and the PFKFB3 inhibitor is intravitreal administration.
[145] the method of any one of [123]-[144], wherein the HIF1-α Pathway Inhibitor and the PFKFB3 inhibitor are administered before the onset of one or more symptoms of AMD.
[146] the method of any one of [123]-[144], wherein the HIF1-α Pathway Inhibitor and the PFKFB3 inhibitor are administered after the onset of one or more symptoms of AMD.
[147] the method of any one of [123]-[144], wherein the HIF1-α Pathway Inhibitor and the PFKFB3 inhibitor are administered during a non-proliferative stage of AMD.
[148] the method of any one of [123]-[144], wherein the HIF1-α Pathway Inhibitor and the PFKFB3 inhibitor are administered during a proliferative stage of AMD.
[149] the method of any one of [123]-[145], wherein treating AMD comprises delaying the onset of AMD.
[150] the method of any one of [123]-[149], wherein one or more symptoms of AMD is reduced in the subject compared to a control subject or compared to the subject prior to treatment with the HIF1-α Pathway Inhibitor and the PFKFB3 inhibitor.
[151] the method of [150], wherein the one or more symptoms of AMD is selected from: choroidal inflammation, choroidal neovascularization, choroidal endothelial cell death, choroidal vascular permeability, choroidal ischemia-reperfusion injury, choroidal leakage area, and occludin disruption, acellular capillary formation, vascular permeability, and occludin disruption.
[152] the method of [150] or [151], wherein the one or more symptoms of AMD are reduced by at least 10%, at least 20%, at least 30%, at least 40%, or at least 50% compared to the control subject or compared to the subject prior to treatment with the HIF1-α Pathway Inhibitor and the PFKFB3 inhibitor.
[153] the method of any one of [123]-[151], wherein one or more vision parameters are increased in the subject compared to the vision parameters a in a control subject or compared to the vision parameters of the subject prior to treatment with the HIF1-α Pathway Inhibitor and the PFKFB3 inhibitor.
[154] the method of [153], wherein the one or more vision parameters are selected from peripheral vision; night vision; color vision; distance vision; close-range vision; and vision clarity;
[155] the method of any one of [123]-[154], wherein the subject has, or is at risk of having wAMD.
[156] the method of any one of [123]-[155], which further comprises administering an anti-VEGF therapeutic agent to the subject.
[157] the method of [156], wherein the anti-VEGF therapeutic agent is bevacizumab, ranibizumab, or aflibercept;
[158] the method of any one of [123]-[157], wherein the subject has received a prior treatment for AMD.
[159] the method of [158], wherein the subject failed to respond to the prior treatment for AMD.
[160] the method of [158] or [159], wherein the prior treatment is a therapeutic procedure selected from a vitrectomy and laser surgery, or a therapeutic agent selected from a steroid and an anti-vascular endothelial growth factor (VEGF) therapy.
[161] the method of [158] or [159], wherein the prior treatment is an anti-vascular endothelial growth factor (VEGF) therapy.
[162] the method of [161], wherein the anti-VEGF therapy is the administration of bevacizumab, ranibizumab, or aflibercept.
[163] a method of treating choroidal neovascularization (CNVM) in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor;
   wherein the PFKFB3 inhibitor does not inhibit PI3K/AKT/mTOR pathway or HIF1-α.
[164] the method of [163], wherein the subject is administered an effective amount of the HIF1-α Pathway Inhibitor and the PFKFB3 inhibitor;
[165] the method of [163], wherein the subject is administered an effective amount of the HIF1-α Pathway Inhibitor and wherein the subject has previously been administered the PFKFB3 Inhibitor;
[166] the method of [163], wherein the subject is administered an effective amount of the PFKFB3 Inhibitor and wherein the subject has previously been administered the HIF1-α Pathway Inhibitor;
[167] the method of any one of [163]-[166], wherein the subject has or is at risk of having CNVM;
[168] the method of any one of [163]-[167], wherein the subject has or has been diagnosed as having CNVM;
[169] the method of any one of [163]-[168], wherein the method of any one of 163(a)-163(c) is administered as a prophylactic treatment for CNVM;
[170] the method of any one of [163]-[169], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor;
[171] the method of any one of [163]-[170], wherein the administered HIF1-α Pathway Inhibitor is silibinin, PX-478 or YC-1.3, or a salt thereof;
[172] the method of any one of [163]-[171], wherein the administered HIF1-α Pathway Inhibitor is ganetespib (ST-9090), phenethyl isothiocyanate, or BAY-87-2243, or a salt thereof;
[173] the method of any one of [163]-[172], wherein the administered HIF1-α Pathway Inhibitor is a HIF1-α Inhibitor.
[174] the method of [173], wherein the HIF1-α Inhibitor is HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor;
[175] the method of [173] or [174], wherein the administered HIF1-α Inhibitor is antisense oligonucleotide EZN-2968 or nanobody AG-1, AG-2, AG-3, AG-4, AG-5, VHH212, or AHPC;
[176] the method of any one of [163]-[175], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[177] the method of any one of [163]-[176], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[178] the method of any one of [163]-[176], wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[179] the method of any one of [163]-[178], wherein the HIF1-α Pathway Inhibitor and the PFKFB3 inhibitor are co-administered to the subject;
[180] the method of any one of [163]-[179], wherein the administration of the HIF1-α Pathway Inhibitor or the PFKFB3 inhibitor is an ocular administration;
[181] the method of any one of [163]-[180], wherein the administration of the HIF1-α Pathway Inhibitor and the PFKFB3 inhibitor is an ocular administration;
[182] the method of [180] or [181], wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration;
[183] the method of any one of [180]-[182], wherein the administration of the HIF1-α Pathway Inhibitor or the PFKFB3 inhibitor is intravitreal administration;
[184] the method of any one of [163]-[183], wherein the administration of the HIF1-α Pathway Inhibitor and the PFKFB3 inhibitor is intravitreal administration;
[185] the method of any one of [163]-[184], wherein the HIF1-α Pathway Inhibitor and the PFKFB3 inhibitor are administered before the onset of one or more symptoms of CNVM;
[186] the method of any one of [163]-[184], wherein the HIF1-α Pathway Inhibitor and the PFKFB3 inhibitor are administered after the onset of one or more symptoms of CNVM;
[187] the method of any one of [163]-[184], wherein the HIF1-α Pathway Inhibitor and the PFKFB3 inhibitor are administered during a non-proliferative stage of CNVM;
[188] the method of any one of [163]-[184] wherein the HIF1-α Pathway Inhibitor and the PFKFB3 inhibitor are administered during a proliferative stage of CNVM;
[189] the method of any one of [163]-[185], wherein treating CNVM comprises delaying the onset of CNVM;
[190] the method of any one of [163]-[189], wherein one or more symptoms of CNVM is reduced in the subject compared to a control subject or compared to the subject prior to treatment with the HIF1-α Pathway Inhibitor and the PFKFB3 inhibitor;
[191] the method of [190], wherein the one or more symptoms of CNVM is selected from: choroidal inflammation, choroidal neovascularization, choroidal endothelial cell death, choroidal vascular permeability, choroidal ischemia-reperfusion injury, choroidal leakage area, and occludin disruption, acellular capillary formation, vascular permeability, and occludin disruption;
[192] the method of [190] or [191], wherein the one or more symptoms of CNVM are reduced by at least 10%, at least 20%, at least 30%, at least 40%, or at least 50% compared to the control subject or compared to the subject prior to treatment with the HIF1-α Pathway Inhibitor and the PFKFB3 inhibitor;
[193] the method of any one of [163]-[192], wherein one or more vision parameters are increased in the subject compared to the vision parameters a in a control subject or compared to the vision parameters of the subject prior to treatment with the HIF1-α Pathway Inhibitor and the PFKFB3 inhibitor;
[194] the method of [193], wherein the one or more vision parameters are selected from peripheral vision; night vision; color vision; distance vision; close-range vision; and vision clarity;
[195] the method of [193], wherein the subject has wet wAMD, histoplasmosis, an eye injury, or myopic macular degeneration;
[196] the method of any one of [163]-[195], which further comprises administering an anti-VEGF therapeutic agent to the subject;
[197] the method of [196], wherein the anti-VEGF therapeutic agent is bevacizumab, ranibizumab, or aflibercept;
[198] the method of any one of [163]-[197], wherein the subject has received a prior treatment for CNVM;
[199] the method of [198], wherein the subject failed to respond to the prior treatment for CNVM;
[200] the method of [198] or [199], wherein the prior treatment is a therapeutic procedure selected from a vitrectomy and laser surgery, or a therapeutic agent selected from a steroid and an anti-vascular endothelial growth factor (VEGF) therapy;
[201] the method of [198] or [199], wherein the prior treatment is an anti-vascular endothelial growth factor (VEGF) therapy; and/or
[202] the method of [201], wherein the anti-VEGF therapy is the administration of bevacizumab, ranibizumab, or aflibercept.

### BRIEF DESCRIPTION OF THE DRAWINGS/FIGURES

FIGS. 1A-1E, depict exemplary PFKFB3 small molecule inhibitors.

### DETAILED DESCRIPTION

### Definitions

Unless otherwise defined, all technical and scientific terms used herein have the same meaning as commonly understood by one of ordinary skill in the art to which this disclosure pertains. Although methods and materials similar or equivalent to those described herein can be used in the practice or testing of the provided compositions, suitable methods and materials are described below. Each publication, patent application, patent, and other reference mentioned herein is herein incorporated by reference in its entirety. In case of conflict, the present specification, including definitions, will control. In addition, the materials, methods, and examples are illustrative only and are not intended to be limiting.

Other features and advantages of the disclosed compositions and methods will be apparent from the following disclosure, drawings, and claims.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the singular form “a”, “an”, and “the”, include plural forms unless it is expressly stated or is unambiguously clear from the context that such is not intended. The singular form “a”, “an”, and “the” also includes the statistical mean composition, characteristics, or size of the particles in a population of particles (e.g., mean polyethylene glycol molecular weight mean liposome diameter, mean liposome zeta potential). The mean particle size and zeta potential of liposomes in a pharmaceutical composition can routinely be measured using methods known in the art, such as dynamic light scattering. The mean amount of a therapeutic agent in a nanoparticle composition may routinely be measured for example, using absorption spectroscopy (e.g., ultraviolet-visible spectroscopy).

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean +/−10% of the recited value. For instance, a nanoparticle composition including a lipid component having about 40% of a given compound may include 30-50% of the compound.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Recitation of ranges of values herein are merely intended to serve as a shorthand method of referring individually to each separate value falling within the range, unless otherwise indicated herein, and each separate value is incorporated into the specification as if it were individually recited herein.

Where embodiments of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed compositions and methods also envisage the explicit exclusion of one or more of any of the group members in the disclosed compositions or methods.

The terms “antibody” and “antigen-binding antibody fragment” and the like, as used herein, include any protein or peptide containing molecule that comprises at least a portion of an immunoglobulin molecule, such as, but not limited to, at least one complementarity determining region (CDR) of a heavy or light chain or an antigen binding portion thereof.

The term “antibody” also includes fragments, specified portions and variants thereof, including antibody mimetics or comprising portions of antibodies that mimic the structure and/or function of an antibody or specified fragment or portion thereof, including single chain antibodies, single binding domain antibodies and antigen binding antibody fragments.

The term “antibody fragment” refers to a portion of an intact antibody, generally the antigen binding or variable region of an intact antibody. Examples of antibody fragments include, but are not limited to Fab, Fab′, F(ab′)2, single chain (scFv) and Fv fragments, diabodies; linear antibodies; single-chain antibody molecules; single Fab arm “one arm” antibodies and multispecific antibodies formed from antibody fragments, among others. Antibody fragments include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an antigen or antigen receptor or binding protein, which can be incorporated into an antibody provided herein.

Antibody fragments can be produced by enzymatic cleavage, synthetic or recombinant techniques, as known in the art. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CHi domain and/or hinge region of the heavy chain. The various portions of antibodies can be joined together chemically by conventional techniques, or can be prepared as a contiguous protein using genetic engineering techniques.

The terms “nucleic acid” and “oligonucleotide” are used interchangeably herein and refer to at least two nucleotides covalently linked together. In some embodiments the HIF1-alpha pathway inhibitor and/or PFKFB3 inhibitor administered according to the provided methods is a therapeutic nucleic acid. In some embodiments, the administered nucleic acid is an ENMD-1198, an shRNA, a Dicer substrate (e.g., dsRNA), an miRNA, an anti-miRNA, an antisense molecule, a decoy, or an aptamer, or a plasmid capable of expressing a ENMD-1198, an shRNA, a Dicer substrate, an miRNA, an anti-miRNA, an antisense molecule, a decoy, or an aptamer.

The nucleic acids administered according to the provided methods are preferably single-stranded or double-stranded and generally contain phosphodiester bonds, although in some cases, nucleic acid/oligonucleotide analogs are included that have alternate backbones, comprising, for example, phosphoramide, phosphorothioate, phosphorodithioate, O-methylphosphoroamidiate linkages, and peptide nucleic acid backbones and linkages. Other analog nucleic acids/oligonucleotides include those with positive backbones; non-ionic backbones, and non-ribose backbones. Nucleic acids/oligonucleotides containing one or more carbocyclic sugars are also included within the definition of nucleic acids and oligonucleotides. These modifications of the ribose-phosphate backbone may be done for example, to facilitate the addition of additional moieties such as labels, or to increase the stability and half-life of such molecules in physiological environments. Nucleic acid/oligonucleotide backbones of oligonucleotides used according to the provided methods can range from about 5 nucleotides to about 750 nucleotides. Preferred nucleic acid/oligonucleotide backbones range from about 5 nucleotides to about 500 nucleotides, and preferably from about 10 nucleotides to about 100 nucleotides in length.

The oligonucleotides administered according to the provided methods are polymeric structures of nucleoside and/or nucleotide monomers capable of specifically hybridizing to at least a region of a nucleic acid target. As indicated above, the “nucleic acids” and “oligonucleotides” used according to the provided methods include, but are not limited to, compounds comprising naturally occurring bases, sugars and intersugar (backbone) linkages, non-naturally occurring modified monomers, or portions thereof (e.g., oligonucleotide analogs or mimetics) which function similarly to their naturally occurring counterpart, and combinations of these naturally occurring and non-naturally occurring monomers. As used herein, the term “modified” or “modification” includes any substitution and/or any change from a starting or natural oligomeric compound, such as an nucleic acid. Modifications to nucleic acids encompass substitutions or changes to internucleoside linkages, sugar moieties, or base moieties, such as those described herein and those otherwise known in the art.

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

“Diabetes mellitus” is a series of dysmetabolic syndromes of carbohydrates, proteins, fats, water, electrolytes and the like that are caused by islet hypofunction, insulin resistance and the like resulting from the effects of genetic factors, immune dysfunction, microbial infections and toxins thereof, free radical toxins, mental factors and other various pathogenic factors on the body, and is mainly characterized by hyperglycemia clinically.

“Diabetic microangiopathy” refers to microangiopathy caused by varying degrees of abnormalities in the microcirculation of various body organs or tissues of diabetics. The process of microangiopathy formation roughly comprises functional changes in microcirculation, endothelial injury, thickening of the basement membrane, increased blood viscosity, aggregation of red blood cells, and adhesion and aggregation of platelets, eventually leading to microthrombosis and/or microvascular occlusion. “Diabetic ocular microangiopathy” refers to ocular microangiopathy caused by diabetes mellitus. “Diabetic retinopathy” includes the diabetes mellitus-induced histological and functional changes of the retina caused by diabetic microangiopathy.

As used herein an “effective amount” refers to a dosage of an agent sufficient to provide a medically desirable result. The effective amount will vary with the desired outcome, the particular disease or condition being treated (or prevented), the age and physical condition of the subject being treated, the severity of the condition, the duration of the treatment, the nature of the concurrent or combination therapy (if any), the specific route of administration and like factors within the knowledge and expertise of the health practitioner. An “effective amount” can be determined empirically and in a routine manner, in relation to the stated purpose.

The terms “subject”, “patient,” “individual,” and “animal” are used interchangeably and refer to mammals such as human patients and non-human primates, as well as experimental animals such as rabbits, rats, and mice, and other laboratory animals. Animals include all vertebrates, e.g., mammals and non-mammals, such as chickens, amphibians, and reptiles. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and other members of the class Mammalia known in the art. In a particular embodiment, the patient is a human.

Terms such as “treating,” or “treatment,” “to treat,” or “therapy,” refer to both (a) therapeutic measures that cure, slow down, attenuate, lessen symptoms of, and/or halt progression of a pathologic condition or disorder and (b) prophylactic or preventative measures that prevent and/or slow the development of a targeted disease or condition. Thus, subjects in need of treatment include those already with the ocular neovascular disorder; those at risk of having the ocular neovascular disorder; and those in whom the ocular neovascular disorder is to be prevented. Subjects are identified as “having or at risk of having” an ocular neovascular disorder or another disorder referred to herein using well-known medical and diagnostic techniques. In certain embodiments, a subject is successfully “treated” according to the provided methods if the subject shows, e.g., total, partial, or transient amelioration or elimination of a symptom associated with the disorder (e.g., diabetic retinopathy, diabetic macular edema, age-related macular degeneration (AMD) and choroidal neovascular membranes). In specific embodiments, the terms “treating,” or “treatment,” “to treat,” or “therapy” refer to the amelioration of at least one measurable physical parameter of an ocular proliferative disorder, such as ocular neovascularization, not necessarily discernible by the patient. In other embodiments, the terms “treating,” or “treatment,” “to treat,” or “therapy,” refer to the inhibition of the progression of an ocular proliferative disorder, either physically by, e.g., stabilization of a discernible symptom, physiologically by, e.g., stabilization of a physical parameter, or both. In other embodiments, the terms “treating,” or “treatment,” “to treat,” or “therapy,” refer to the reduce the alleviation of symptoms, the reduction of inflammation, the inhibition of cell death, and/or the restoration of cell function can be with the HIF1-α Pathway Inhibitor and PFKFB3 inhibitor compositions disclosed herein, or in further combination with an additional Therapeutic agent.

The term “pharmaceutically acceptable carrier” refers to an ingredient in a pharmaceutical formulation, other than an active ingredient, which is nontoxic to a subject. A pharmaceutically acceptable carrier includes, but is not limited to, a buffer, carrier, excipient, stabilizer, diluent, or preservative. Pharmaceutically acceptable carriers can include for example, one or more compatible solid or liquid filler, diluents or encapsulating substances which are suitable for administration to a human or other subject.

“Therapeutic agent” the therapeutic agent or therapeutic agents used according to the disclosed compositions and methods can include any agent directed to treat a condition in a subject. Examples of therapeutic agents that may be suitable for use in accordance with the provided methods include HIF1-α Pathway Inhibitors, PFKFB3 Inhibitors, anti-VEGF therapeutic agents (e.g., anti-VEGF antibody (e.g., bevacizumab and ranibizumab), and small molecule VEGF receptor inhibitors (e.g., sunitinib, sorafenib and pazopanib). corticosteroids (e.g., hydrocortisone or a glucocorticoid such as, cortisone, ethamethasoneb, prednisone, prednisolone, triamcinolone, dexamethasone and methylprednisolone). “Therapeutic agents” also refer to salts, acids, and free based forms of the above agents.

### PFKFB3 Inhibitors

PFKFB3 (6-phosphofructo-2-kinase-fructose-2,6-bisphosphatase 3) is a bifunctional protein that is involved in both the synthesis and degradation of fructose-2,6-bisphosphate, a regulatory molecule that controls glycolysis in eukaryotes and is required for cell cycle progression and the prevention of apoptosis.

In some embodiments, the disclosure provides a method of treating an ocular neovascular disease or condition in a subject in need thereof that comprises:
(a) administering an effective amount of a HIF1-α Pathway Inhibitor or a HIF1-α Inhibitor and an PFKFB3 inhibitor to the subject;
(b) administering an effective amount of a HIF1-α Pathway Inhibitor or a HIF1-α Inhibitor to the subject, wherein the subject has previously been administered a PFKFB3 Inhibitor; or
(c) administering an effective amount of a PFKFB3 Inhibitor to the subject wherein the subject has previously been administered a HIF1-α Pathway Inhibitor or a HIF1-α Inhibitor to the subject;
wherein the PFKFB3 Inhibitor does not inhibit the PI3K/AKT/mTOR pathway or HIF1-α.

In some embodiments, the disclosure provides the method according to any one of (a)-(c) wherein PFKFB3 is upregulated in the subject.

In some embodiments, the disclosure provides the method according to any one of (a)-(c) wherein HIF1-α is upregulated in the subject.

In some embodiments, the disclosure provides the method according to any one of (a)-(c) wherein PFKFB3 and HIF1-α is upregulated in the subject.

In some embodiments, the disclosure provides the method according to any one of (a)-(c) wherein PFKFB3 is upregulated independent of HIF1-α in the subject.

In some embodiments, the disclosure provides the method according to any one of (a)-(c) wherein HIF1-α is upregulated independent of PFKFB3 in the subject.

In some embodiments, the disclosure provides a method of treating an ocular neovascular disease or condition in a subject in need thereof that comprises administering an effective amount of a PFKFB3 inhibitor to the subject. In particular embodiments, the a PFKFB3 inhibitor is administered in combination with a VEGF antagonist.

In further embodiments, the treated ocular neovascular disease or condition is diabetic retinopathy (DR), diabetic macular edema (DME), age-related macular degeneration (AMD), such as wet AMD (wAMD), or choroidal neovascular membranes.

The PFKFB3 Inhibitors that can be used according to the provided methods are not particularly limited. In some embodiments, the administered PFKFB3 Inhibitor is an antibody or a PFKFB3-binding antibody (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, miRNA, dsRNA, ssRNA, and shRNA), a peptibody, a nanobody, a PFKFB3 inhibitory binding polypeptide, or a small molecule PFKFB3 Inhibitor.

In some embodiments, the PFKFB3 inhibitor administered according to the provide methods has an IC50 for a PFKFB3 activity/function of 100 μM or lower concentration for a PFKFB3 activity. In some embodiments, the PFKFB3 inhibitor has an IC50 of at least or at most or about 200, 100, 80, 50, 40, 20, 10, 5, or 1 μM, or at least or at most or about 100, 10, or 1 nM, or lower (or any range or value derivable therefrom). In some embodiments, the PFKFB3 inhibitor inhibits the expression of PFKFB3. Assays for determining the ability of a compound to inhibit PFKFB3 activity are known in the art. In some embodiments, the inhibition of PFKFB3 activity or expression is a decrease as compared with a control level or sample. In some embodiments, a functional assay such as an MTT assay, cell proliferation assay, BRDU or Ki67 immunofluorescence assay, apoptosis assay, or glycolysis assay is used to assay for the ability of a composition to inhibit PFKFB3 activity.

In some embodiments, the PFKFB3 Inhibitor administered according to the provided methods is an antibody or a PFKFB3-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), In particular embodiments, the administered PFKFB3 Inhibitor is a nanobody (e.g., a VHH).

In some embodiments, the HIF1-A Inhibitor administered according to the provided methods is a therapeutic nucleic acid. In some embodiments, the therapeutic nucleic acid is an aptamer, antisense molecule, ribozyme, a Dicer substrate, miRNA, dsRNA, ssRNA, and shRNA). In particular embodiments, the HIF1-ac Inhibitor administered according to the provided methods is an siRNA or an antisense oligonucleotide.

Representative examples of human PFKFB3 coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit PFKFB3 activity can routinely be designed and prepared based on each of the above human PFKFB3 transcript sequences using methods known in the art.

The administration of PFKFB3 inhibitory nucleic acids or any ways of inhibiting gene expression of PFKFB3 known in the art are contemplated in certain embodiments of the provided methods. Examples of inhibitory nucleic acid include but are not limited to, antisense nucleic acids such as: ENMD-1198 (small interfering RNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of PFKFB3 or prevent the translation of a PFKFB3 gene transcript in a cell. In some embodiments, the PFKFB3 inhibitory nucleic acid administered according to the provided methods is from 16 to 1000 nucleotides in length. In certain embodiments the administered PFKFB3 inhibitory nucleic acid is from 18 to 100 nucleotides long. In certain embodiments the administered PFKFB3 inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the PFKFB3 inhibitory nucleic acid administered according to the provided methods is capable of decreasing the expression of PFKFB3 by at least 10%, 20%, 30%, or 40%, more particularly by at least 50%, 60%, or 70%, and most particularly by at least 75%, 80%, 90%, 95% or more or any range or value in between the foregoing.

In some embodiments, the PFKFB3 inhibitory nucleic acid administered according to the provided methods is between 17 to 25 nucleotides in length and comprises a 5′ to 3′ sequence that is at least 90% complementary to the 5′ to 3′ sequence of a mature PFKFB3 mRNA (e.g., a sequence as disclosed in any one or more of GenBank accession nos. NM_004566.3, NM_001145443.2, NM_001282630.2, NM_001314063.1, NM_001323016.1, NM_001323017.1, and NM_001363545.2). In some embodiments, the administered PFKFB3 inhibitory nucleic acid is 17, 18, 19, 20, 21, 22, 23, 24, or 25 nucleotides in length, or any range derivable therein. In some embodiments, the administered PFKFB3 inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature PFKFB3 mRNA (e.g., a sequence as disclosed in any one or more of GenBank accession nos. NM_004566.3, NM_001145443.2, NM_001282630.2, NM_001314063.1, NM_001323016.1, NM_001323017.1, and NM_001363545.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the PFKFB3 inhibitory nucleic acid administered according to the provided methods is a miRNA. In further embodiments, the administered miRNA is a member selected from: hsa-mir-26b-5p (MIRT028775), hsa-mir-330-3p (MIRT043840), hsa-mir-6779-5p (MIRT454747), hsa-mir-6780a-5p (MIRT454748), hsa-mir-3689c (MIRT454749), hsa-mir-3689b-3p (MIRT454750), hsa-mir-3689a-3p (MIRT454751), hsa-mir-30b-3p (MIRT454752), hsa-mir-1273h-5p (MIRT454753), hsa-mir-6778-5p (MIRT454754), hsa-mir-1233-5p (MIRT454755), hsa-mir-6799-5p (MIRT454756), hsa-mir-7106-5p (MIRT454757), hsa-mir-6775-3p (MIRT454758), hsa-mir-1291 (MIRT454759), hsa-mir-765 (MIRT454760), hsa-mir-423-5p (MIRT454761), hsa-mir-3184-5p (MIRT454762), hsa-mir-6856-5p (MIRT454763), hsa-mir-6758-5p (MIRT454764), hsa-mir-3185 (MIRT527973), hsa-mir-6892-3p (MIRT527974), hsa-mir-6840-5p (MIRT527975), and hsa-mir-6865-3p (MIRT527976).

In some embodiments, the PFKFB3 inhibitor administered according to the provide methods is a small molecule. The administered small molecule PFKFB3 inhibitors may be any small molecules that is determined to inhibit PFKFB3 function or activity. Such small molecules may be determined based on functional assays in vitro or in vivo. In some embodiments, the PFKFB3 inhibitor small molecules administered according to the provide methods is a small molecule PFKFB3 inhibitory molecules disclosed in U.S. publication nos. 20130059879, 20120177749, 20100267815, 20100267815, and 20090074884, the disclosure of each of which is herein incorporated by reference in its entirety.

In some embodiments, the PFKFB3 inhibitor administered according to the provided methods is at least one of: (1H-Benzo[g]indol-2-yl)-phenyl-methanone; (3H-Benzo[e]indol-2-yl)-phenyl-methanone; (3H-Benzo[e]indol-2-yl)-(4-methoxy-phenyl)-methanone; (3H-Benzo[e]indol-2-yl)-pyridin-4-yl-methanone; HCl salt of (3H-Benzo[e]-indol-2-yl)-pyridin-4-yl-methanone; (3H-Benzo[e]indol-2-yl)-(3-methoxy-phenyl)-methanone; (3H-Benzo[e]indol-2-yl)-pyridin-3-yl-methanone; (3H-Benzo[e]ind-ol-2-yl)-(2-methoxy-phenyl)-methanone; (3H-Benzo[e]indol-2-yl)-(2-hydroxy-phenyl)-methanone; (3H-Benzo[e]indol-2-yl)-(4-hydroxy-phenyl)-methanone; (5-Methyl-3H-benzo[e]indol-2-yl)-phenyl-methanone; Phenyl-(7H-pyrrolo[2,3-h]quinolin-8-yl)-methanone; (3H-Benzo[e]indol-2-yl)-(3-hydroxy-phenyl)-methanone; (3H-benzo[e]indol-2-yl)-(2-chloro-pyridin-4-yl)-methanone; (3H-benzo[e]indol-2-yl)-(1-oxy-pyridin-4-yl)-methanone; Phenyl-(6,7,8,9-tetrahydro-3H-benzo[e]indol-2-yl)-methanone; (3H-Benzo[e]indol-2-yl)-(4-hydroxy-3-methoxylthenyl)-methanone; (3H-Benzo[e]indol-2-yl)-(4-benzyloxy-3-methoxy-phenyl)-methanone; 4-(3H-Benzo[e]indole-2-carbonyl)-benzoic acid methyl ester; 4-(3H-Benzo[e]indole-2-carbonyl)-benzoic acid; (4-Amino-phenyl)-(3H-benzo[e]indol-2-yl)-methanone; 5-(3H-Benzo[e]indole-2-carbonyl)-2-benzyloxy-benzoic acid methyl; 5-(3H-Benzo[e]indole-2-carbonyl)-2-benzyloxy-benzoic Acidmethanone; (3H-Benzo[e]indol-2-yl)-(2-methoxy-pyridin-4-yl)-methanone; (5-Fluoro-3H-benzo[e]-indol-2-yl)-(3-methoxy-phenyl)-methanone; (5-Fluoro-3H-benzo[e]indol-2-yl)-pyridin-4-yl-methanone; (4-Benzyloxy-3-methoxy-phenyl)-(5-fluoro-3H-benzo[e]indol-2-yl)-methanone; (5-Fluoro-3H-benzo[e]indol-2-yl)-(4-hydroxy-3-methoxy-phenyl)-methanone; (3H-Benzo[e]indol-2-yl)-(3-hydroxymethyl-phenyl)-methanone; Cyclohexyl-(5-fluoro-3H-benzo[e]indol-2-yl)-methanone; (5-Fluoro-3H-benzo[e]indol-2-yl)-(3-fluoro-4-hydroxy-phenyl)-methanone; (3H-Benzo[e]indol-2-yl)-p-tolyl-methanone; (3H-Benzo-[e]indol-2-yl)-(3-methoxy-phenyl-methanol; (3H-Benzo[e]indol-2-yl)-pyridin-4-yl-methanol; 3H-Benzo[e]indole-2-carboxylic acid phenylamide; 3H-Benzo[e]indole-2-carboxylic acid (3-methoxy-phenyl)-amide; (3H-Benzo[e]indol-2-yl)-(4-dimethylamino-phenyl)-methanone; (4-Amino-3-methoxy-phenyl)-(3H-benzo[e]indol-2-yl)-methanone; (4-Amino-3-methoxy-phenyl)-(5-hydroxy-3H-benzo[e]indol-2-yl)-methanone; (4-Amino-3-methoxy-phenyl)-(5-methoxy-3H-benzo[e]indol-2-yl)-methanone; N-[4-(3H-Benzo[e]indole-2-carbonyl)-phenyl]-methanesulfonamide; 3H-Benzo[e]indole-2-carboxylic acid (4-amino-phenyl)-amide; (4-Amino-phenyl)-(5-methoxy-3H-benzo[e]-indol-2-yl)-methanone; (4-Amino-2-fluoro-phenyl)-(5-methoxy-3H-benzo[e]indol-2-yl)-methanone; (4-Amino-3-fluoro-phenyl)-(5-methoxy-3H-benzo[e]indol-2-yl)-methanone; (4-Amino-2-methoxy-phenyl)-(5-methoxy-3H-benzo[e]indol-2-yl)-methanone; (4-Amino-phenyl)-(9-methoxy-3H-benzo[e]indol-2-yl)-methanone; (4-Amino-3-methoxy-phenyl)-(9-meth-oxy-3H-benzo[e]indol-2-yl)-methanone; (4-Amino-2-methoxy-phenyl)-(9-methoxy-3H-benzo[e]indol-2-yl)-methanone; (4-Amino-3-fluoro-phenyl)-(9-methoxy-3H-benzo[e]-indol-2-yl)-methanone; (4-Amino-2-fluoro-phenyl)-(9-methoxy-3H-benzo[e]indol-2-yl)-methanone; (4-Amino-3-fluoro-phenyl)-(3H-benzo[e]indol-2-yl)-methanone; (4-Amino-2-fluoro-phenyl)-(3H-benzo[e]indol-2-yl)-methanone; (4-Amino-phenyl)-(7-methoxy-3H-benzo[e]indol-2-yl)-methanone; (4-Amino-phenyl)-(5-hydroxy-3-methyl-3H-benzo[e]indol-2-yl)-methanone; (7-Amino-5-fluoro-9-hydroxy-3H-benzo[e]indol-2-yl)-(3-methyl-pyridin-4-yl)-methanone; (5-Amino-3H-pyrrolo[3,2-f]isoquinolin-2-yl)-(3-methoxy-pyridin-4-yl)-methanone; (4-Amino-2-methyl-phenyl)-(9-hydroxy-3H-pyrrolo[2,3-c]quinolin-2-yl)-methanone; and (4-Amino-phenyl)-(7-methanesulfonyl-3H-benzo[e]indol-2-yl)-methanone, or a salt thereof.

In some embodiments, the PFKFB3 inhibitor administered according to the provided methods is at least one of: 1-Pyridin-4-yl-3-quinolin-4-yl-propenone; 1-Pyridin-4-yl-3-quinolin-3-yl-propenone; 1-Pyridin-3-yl-3-quinolin-2-yl-propenone; 1-Pyridin-3-yl-3-quinolin-4-yl-propenone; l-Pyridin-3-yl-3-quinolin-3-yl-propenone; l-Naphthalen-2-yl-3-quinolin-2-yl-propenone; l-Naphthalen-2-yl-3-quinolin-3-yl-propenone; l-Pyridin-4-yl-3-quinolin-3-yl-propenone; 3-(4-Hydroxy-quinolin-2-yl)-1-pyridin-4-yl-propenone; 3-(8-Hydroxy-quinolin-2-yl)-1-pyridin-3-yl-propenone; 3-Quinolin-2-yl-1-p-tolyl-propenone; 3-(8-Hydroxy-quinolin-2-yl)-1-pyridin-4-yl-propenone; 3-(8-Hydroxy-quinolin-2-yl)-1-p-tolyl-propenone; 3-(4-Hydroxy-quinolin-2-yl)-1-p-tolyl-propenone; 1-Phenyl-3-quinol-in-2-yl-propenone; 1-Pyridin-2-yl-3-quinolin-2-yl-propenone; 1-(2-Hydroxy-phenyl)-3-quinolin-2-yl-propenone; 1-(4-Hydroxy-phenyl)-3-quinolin-2-yl-propenone; 1-(2-Amino-phenyl)-3-quinolin-2-yl-propenone; 1-(4-Amino-phenyl)-3-quinolin-2-yl-propenone; or a salt thereof.

In some embodiments, the PFKFB3 inhibitor administered according to the provided methods is at least one of: 4-(3-Quinolin-2-yl-acryloyl)-benzamide; 4-(3-Quinolin-2-yl-acryloyl)-benzoic acid; 3-(8-Methyl-quinolin-2-yl)-1-pyridin-4-yl-propenone; 1-(2-Fluoro-pyridin-4-yl)-3-quinolin-2-yl-propenone; 3-(8-Fluoro-quinolin-2-yl)-1-pyridin-4-yl-propenone; 3-(6-Hydroxy-quinolin-2-yl)-1-pyridin-4-yl-propenone; 3-(8-Methylamino-quin-olin-2-yl)-1-pyridin-4-yl-propenone; 3-(7-Methyl-quinolin-2-yl)-1-pyridin-4-yl-propenone; and 1-Methyl-4-[3-(8-methyl-quinolin-2-yl)-acryloyl]-pyridinium, or a salt thereof.

In some embodiments, the PFKFB3 inhibitor administered according to the provided methods is at least one of: PFK15 (1-(4-pyridinyl)-3-(2-quinolinyl)-2-propen-1-one); (2S)—N-[4-[[3-Cyano-1-(2-methyl-propyl)-1H-indol-5-yl]oxy]phenyl]-2-pyrrolidine-carboxamide 3PO (3-(3-Pyridinyl)-1-(4-pyridinyl)-2-propen-1-one); (2S)—N-[4-[[3-Cyano-1-[(3,5-dimethyl-4-isoxazolyl)methyl]-1H-indol-5-yl]oxy]phenyl]-2-pyrrolidine-carboxamide; and Ethyl 7-hydroxy-2-oxo-2H-1-benzopyran-3-carboxylate, or a salt thereof.

In a particular embodiment, the PFKFB3 inhibitor administered according to the provided methods is PFK15, or a salt thereof.

In a particular embodiment, the PFKFB3 inhibitor administered according to the provided methods is PFK158 ((E)-1-(4-Pyridinyl)-3-[7-(trifluoromethyl)-2-quinolinyl]-2-propen-1-one), or a salt thereof.

In a particular embodiment, the PFKFB3 inhibitor administered according to the provided methods is BrAcNHEtOP (N-bromoacetylethanolamine phosphate), or a salt thereof.

In a particular embodiment, the PFKFB3 inhibitor administered according to the provided methods is AZ67, or a salt thereof.

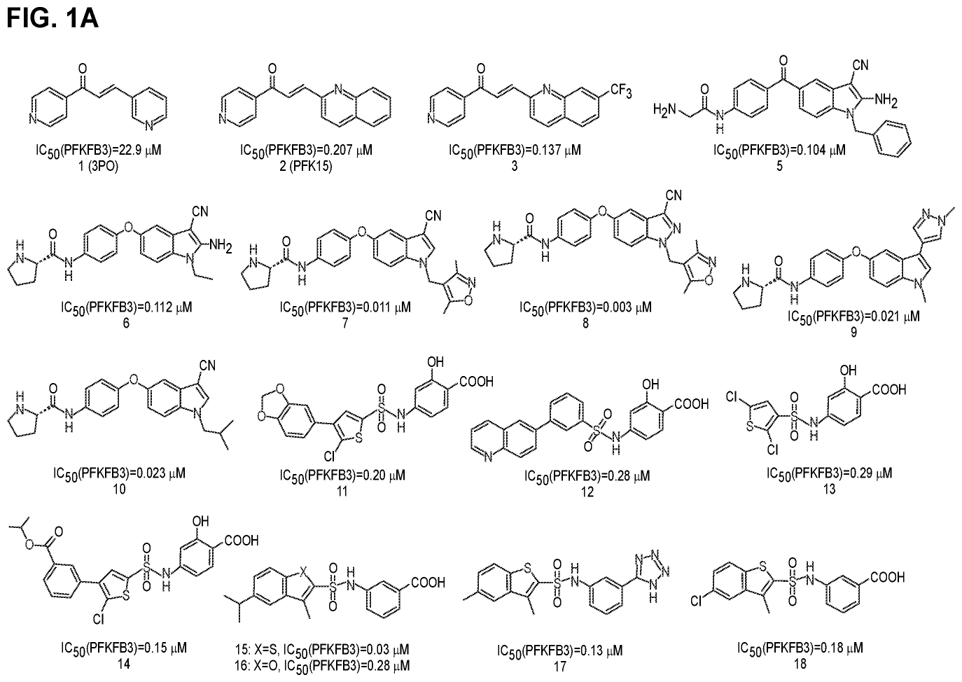

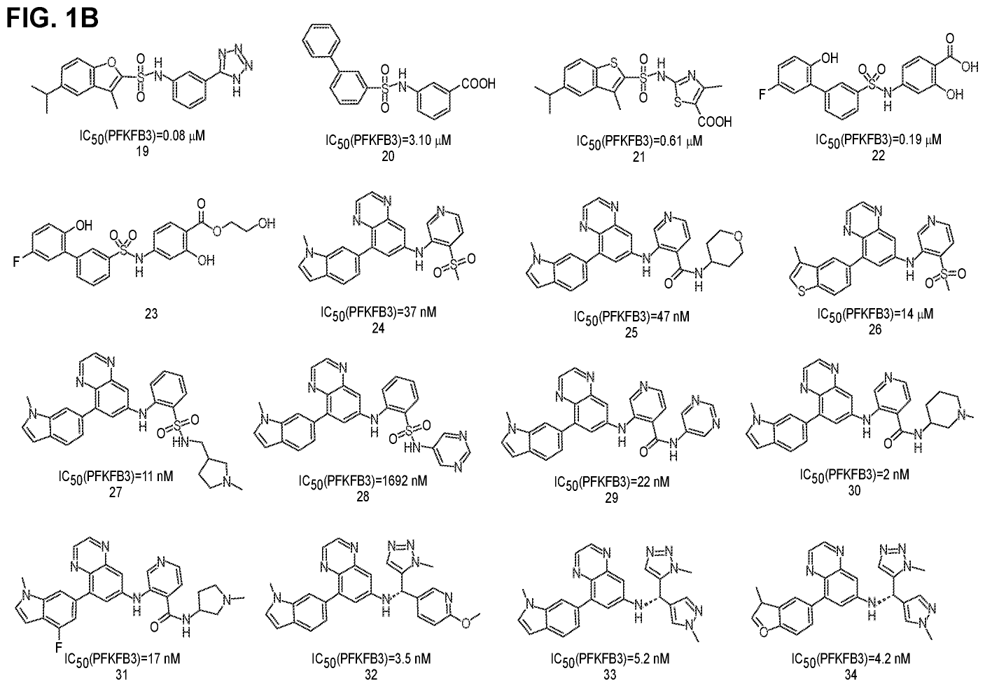

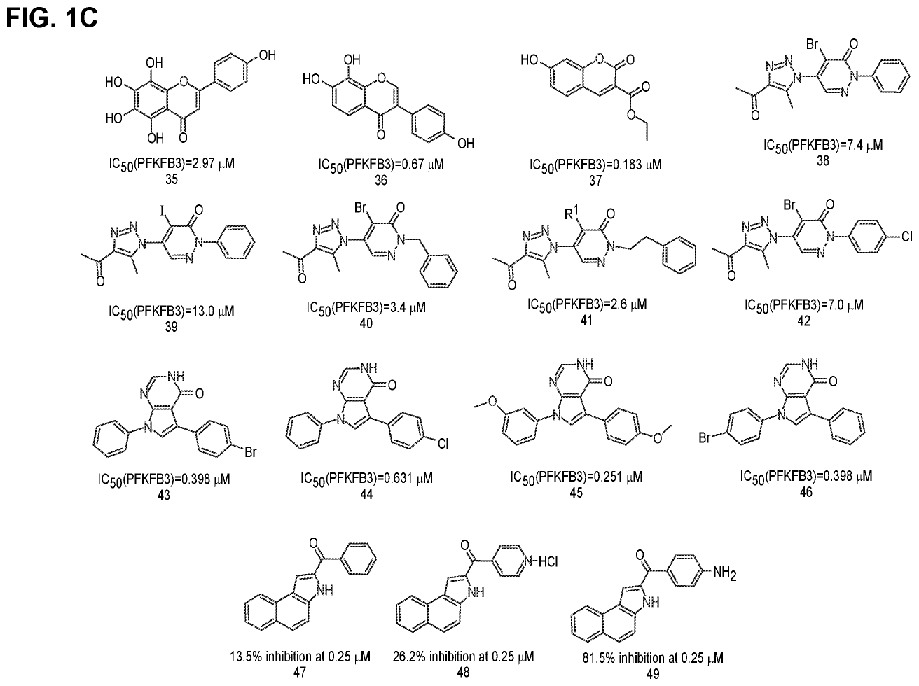

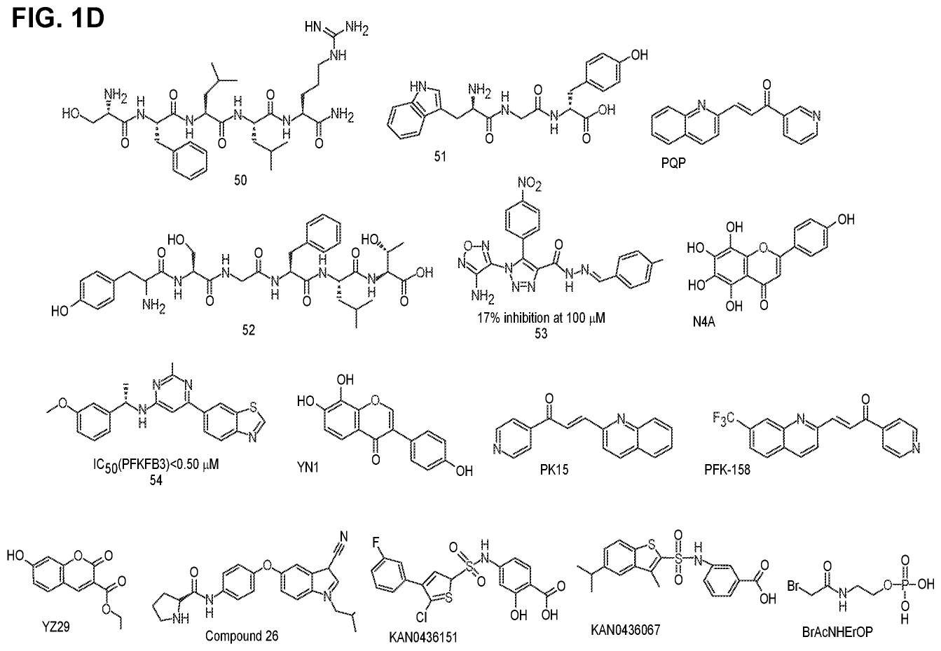

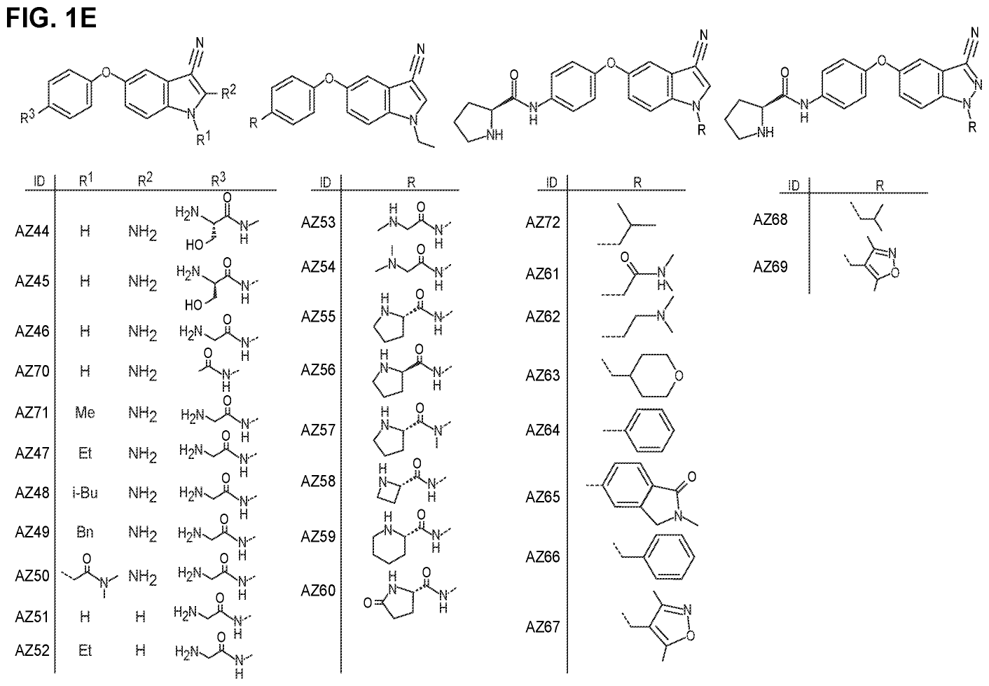

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In a particular embodiment, the PFKFB3 inhibitor administered according to the provided methods is KAN0436151, or a salt thereof.

In a particular embodiment, the PFKFB3 inhibitor administered according to the provided methods is KAN0436067, or a salt thereof.

### HIF1-α Pathway Inhibitors

Hypoxia-inducible factor 1-alpha (HIF-1-alpha) is a subunit of a heterodimeric transcription factor hypoxia-inducible factor 1 (HIF-1) that is considered to be the master transcriptional regulator of cellular and developmental response to hypoxia.

In some embodiments, the disclosure provides a method of treating an ocular neovascular disease or condition in a subject in need thereof that comprises:
(a) administering an effective amount of a HIF1-α Pathway Inhibitor or a HIF1-α Inhibitor and an PFKFB3 inhibitor to the subject;
(b) administering an effective amount of a HIF1-α Pathway Inhibitor or a HIF1-α Inhibitor to the subject, wherein the subject has previously been administered a PFKFB3 Inhibitor; or
(c) administering an effective amount of a PFKFB3 Inhibitor to the subject wherein the subject has previously been administered a HIF1-α Pathway Inhibitor or a HIF1-α Inhibitor; and
wherein the PFKFB3 inhibitor does not inhibit PI3K/AKT/mTOR pathway or HIF1-α.

In some embodiments, the disclosure provides the method according to any one of (a)-(c) wherein HIF1-α is upregulated in the subject.

In some embodiments, the disclosure provides the method according to any one of (a)-(c) wherein PFKFB3 is upregulated in the subject.

In some embodiments, the disclosure provides the method according to any one of (a)-(c) wherein PFKFB3 and HIF1-α is upregulated in the subject.

In some embodiments, the disclosure provides the method according to any one of (a)-(c) wherein PFKFB3 is upregulated independent of HIF1-α in the subject.

In some embodiments, the disclosure provides the method according to any one of (a)-(c) wherein HIF1-α is upregulated independent of PFKFB3 in the subject.

In some embodiments, the disclosure provides a method of treating an ocular neovascular disease or condition in a subject in need thereof that comprises administering an effective amount of a HIF1-α Pathway Inhibitor or a HIF1-α Inhibitor to the subject. In particular embodiments the effective amount downregulates VEGF and PFKFB3. In particular embodiments, the effective amount reduces vascular and/or neuronal impairment in the subject.

In further embodiments, the treated ocular neovascular disease or condition is diabetic retinopathy (DR), diabetic macular edema (DME), age-related macular degeneration (AMD), such as wet AMD (wAMD), or choroidal neovascular membranes.

The term “HIF1-α Pathway-α Inhibitor” as used herein refers to a composition that inhibits or reduces HIF1-α directly or indirectly via inhibiting one or more activities of the PI3K/AKT/mTOR pathway that is upstream of the HIF1-α pathway. The term “HIF1-α Inhibitor” is used herein to refer to a composition that inhibits or reduces HIF1-α directly. Thus, for example, mTOR pathway inhibitors such as temsirolimus, everolimus, and sirolimus are considered herein to be “HIF1-α Pathway-α Inhibitors”, but not “HIF1-α Inhibitors.”

The “HIF1-α Pathway-α Inhibitors” that can be administered according to the provided methods are not particularly limited. In some embodiments, the administered HIF1-α Pathway Inhibitor is an antibody or a HIF1-α-binding antibody fragment (e.g., a single chain antibody, a single-domain antibody ((e.g., VHH AG-1, AG-2, AG-3, AG-4, or AG-5, AHPC, or VHH212), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor

In some embodiments, the administered HIF1-α Pathway Inhibitor administered according to the provided methods has an IC50 for a HIF1-α activity/function of 100 μM or lower concentration for a HIF1-α activity. In some embodiments, the HIF1-α Pathway Inhibitor has an IC50 of at least or at most or about 200, 100, 80, 50, 40, 20, 10, 5, or 1 M, or at least or at most or about 100, 10, or 1 nM, or lower (or any range or value derivable therefrom). In some embodiments, the HIF1-α Pathway Inhibitor inhibits the expression of HIF1-α. Assays for determining the ability of a compound to inhibit HIF1-α activity are known in the art. In some embodiments, the inhibition of HIF1-α activity or expression is a decrease as compared with a control level or sample. In some embodiments, a functional assay such as an MTT assay, cell proliferation assay, BRDU or Ki67 immunofluorescence assay, apoptosis assay, or glycolysis assay is used to assay for the ability of a composition to inhibit HIF1-α activity.

The HIF1-α Inhibitors that can be administered according to the provided methods are not particularly limited. In some embodiments, the HIF1-α Inhibitor modulates one or more of HIF-1α mRNA expression; HIF-1α protein translation or degradation; HIF-1α/HIF-10 dimerization; HIF-1α-DNA binding (e.g., HIF-1α/HRE); and/or HIF-1α transcriptional activity (e.g., CH-1 of p300/C-TAD of HIF-1α).

In some embodiments, the HIF1-α Inhibitor administered according to the provided methods is a small molecule. In some embodiments, the HIF1-α Inhibitor administered according to the provided methods is a protein or polypeptide (e.g., an anti HIF1 antibody or antibody fragment that binds HIF1). In some embodiments, the HIF1-α Inhibitor administered according to the provided methods is a therapeutic nucleic acid (e.g., an aptamer, antisense molecule, ribozyme, a Dicer substrate, siRNA, miRNA, dsRNA, ssRNA, or an shRNA).

In some embodiments, the HIF1-α Pathway Inhibitor administered according to the provided methods is a HIF1-α Pathway Inhibitor (e.g., a PI3K pathway inhibitor, a MAPK pathway inhibitor, an Akt pathway inhibitor, and/or an mTOR inhibitor); a HIF translation inhibitor (e.g., a topoisomerase inhibitor, a microtubule targeting drug a cardiac glycoside, or an antisense HIF-1a mRNA); an inhibitor of HIF stability, nuclear localization or dimerization (e.g., acriflavine or an HDAC inhibitor); an inhibitor of HIF transactivation (e.g., a HIF1 coactivator recruitment inhibitor or a HIF1 DNA binding inhibitor).

In some embodiments, the HIF1-α Inhibitor administered according to the provided methods is a HIF1-α Pathway Inhibitor (e.g., a PI3K pathway inhibitor, a MAPK pathway inhibitor, an Akt pathway inhibitor, and/or an mTOR inhibitor). In some embodiments, the HIF1-α Inhibitor administered according to the provided methods is a PI3K pathway inhibitor. In one embodiment, the administered HIF1-α Pathway Inhibitor is P3155, LY29, LY294002, wortmannin, or GDC-0941. In one embodiment, the administered HIF1-α Pathway Inhibitor is resveratrol. In another embodiment, the administered HIF1-α Pathway Inhibitor is a glyceolin. In some embodiments, the HIF1-α Pathway Inhibitor administered according to the provided methods is an mTOR inhibitor. In one embodiment, the administered HIF1-α Pathway Inhibitor is rapamycin, temsirolimus (CC1-779), everolimus, sirolimus, or PP242.

In a particular embodiment, the administered HIF1-α Inhibitor is silibinin.

In some embodiments, the HIF1-α Inhibitor administered according to the provided methods is a HIF translation inhibitor. In one embodiment, the administered HIF1-α Inhibitor is PX-478 (S-2-amino-3-[4′-N,N-bis(chloroethyl)[amino]phenyl propionic acid N-oxide dihydrochloride), NSC-64421, camptothecin (CPT), SN38, irinotecan, topotecan, NSC-644221, cycloheximide, or apigenin, or a salt thereof. In one embodiment, the administered HIF1-α Inhibitor is aminoflavone, KC7F2 (N,N′-(disulfanediylbis(ethane-2,1-diyl))bis(2,5-dichlorobenzene-sulfonamide), 2-meth-oxyestra-diol (2ME2) or an analog or salt thereof. In one embodiment, the administered HIF1-α Inhibitor is ENMD-1198, ENMD-1200, or ENMD-1237, or a salt thereof. In one embodiment, the administered HIF1-α Inhibitor is EZN-2208, or a salt thereof.

In a particular embodiment, the administered HIF1-α Inhibitor is PX-478, or a salt thereof.

In some embodiments, the HIF1-α Inhibitor administered according to the provided methods is a cardiac glycoside. In one embodiment, the administered cardiac glycoside is digoxin, or a salt thereof. In another embodiment, the administered cardiac glycoside ouabain or proscillardin A, or a salt thereof.

In some embodiments, the HIF1-α Pathway Inhibitor administered according to the provided methods is a topoisomerase inhibitor. In one embodiment, the administered topoisomerase inhibitor is camptothecin (CPT), SN38, irinotecan, or topotecan (e.g., PEG-SN38), or a salt thereof.

In some embodiments, the HIF1-α Pathway Inhibitor administered according to the provided methods is a microtubule targeting drug. In one embodiment, the administered microtubule targeting drug is 2 methoxyestradiol (2ME2), ENMD-1198, ENMD-1200, ENMD-1237, or Taxotere, or a salt thereof.

In some embodiments, the HIF1-α Inhibitor administered according to the provided methods is a therapeutic nucleic acid. In some embodiments therapeutic nucleic acid is an aptamer, antisense molecule, ribozyme, a Dicer substrate, siRNA, miRNA, dsRNA, ssRNA, and shRNA). In some embodiments, therapeutic nucleic acid is an antisense oligonucleotide.

In some embodiments, the HIF1-A Inhibitor administered according to the provided methods is a siRNA or an antisense oligonucleotide. In one embodiment, the administered HIF1-α Inhibitor is EZN-2968. In one embodiment, the administered HIF1-α Inhibitor is RX-0047.

Representative examples of human HIF1-A coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit HIF1-A activity can routinely be designed and prepared based on each of the above human HIF1-A transcript sequences using methods known in the art.

The administration of HIF1-A inhibitory nucleic acids or any ways of inhibiting gene expression of HIF1-A known in the art are contemplated in certain embodiments of the provided methods. Examples of inhibitory (therapeutic) nucleic acid include but are not limited to, antisense nucleic acids such as: ENMD-1198 (small interfering RNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of HIF1-A or prevent the translation of a HIF1-A gene transcript in a cell. In some embodiments, the HIF1-A inhibitory nucleic acid administered according to the provided methods is from 16 to 1000 nucleotides in length. In certain embodiments the administered HIF1-A inhibitory nucleic acid is from 18 to 100 nucleotides long. In certain embodiments the administered HIF1-A inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the HIF1-A inhibitory nucleic acid administered according to the provided methods is capable of decreasing the expression of HIF1-A by at least 10%, 20%, 30%, or 40%, more particularly by at least 50%, 60%, or 70%, and most particularly by at least 75%, 80%, 90%, 95% or more or any range or value in between the foregoing.

In some embodiments, the HIF1-A inhibitory nucleic acid administered according to the provided methods is between 17 to 25 nucleotides in length and comprises a 5′ to 3′ sequence that is at least 90% complementary to the 5′ to 3′ sequence of a mature HIF1-A mRNA (e.g., as disclosed in any one or more of GenBank accession nos. NM_001530.4, NM_181054.3, and NM_001243084.2). In some embodiments, the administered HIF1-A inhibitory nucleic acid is 17, 18, 19, 20, 21, 22, 23, 24, or 25 nucleotides in length, or any range derivable therein. In some embodiments, the administered HIF1-A inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature HIF1-A mRNA (e.g., as disclosed in any one or more of GenBank accession nos. NM_001530.4, NM_181054.3, and NM_001243084.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the HIF1-A inhibitory nucleic acid administered according to the provided methods is a miRNA mimic. In some embodiments, the administered HIF1-α Inhibitor is a miR-483 mimic.

In some embodiments, the HIF1-α Inhibitor administered according to the provided methods is an inhibitor of HIF stability, nuclear localization or dimerization. In one embodiment, the inhibitor administered according to the provided methods destabilizes HIF. In one embodiment, the inhibitor administered according to the provided methods is a histone deacetylase inhibitor (HDACI). In a further embodiment, the administered HDACI is LW6/CAY10585, vorinostat, romidepsin (FK228), panobinostat, belinostat, Trichostatin A (TSA), LAQ824, or phenethyl isothiocyanate, or a salt thereof. In one embodiment, the inhibitor administered according to the provided methods is PX-12/pleurotin, HIF-1a inhibitor (CAS No. 934593-90-5), cryptotanshinone, or BAY 87-2243 (1-cyclopropyl-4-[4-[[5-methyl-3-[3-[4-(trifluoromethoxy) phenyl]-1,2,4-oxadiazol-5-yl]-1H-pyrazol-1-yl]methyl]-2-pyridinyl]-piperazine), or a salt thereof. In one embodiment, the inhibitor administered according to the provided methods is IDF-11774, Bisphenol A/Dimethyl bisphenol A, or a salt thereof. Chrysin (5,7-dihydroxy-flavone), or SCH66336, or a salt thereof. In one embodiment, the inhibitor administered according to the provided methods is geldanamycin or analog thereof, 17-AAG (tanespimycin: allylamino-17-demethoxygeldanamycin), 17-DMAG (alvespimycin), 17AG, radiccicol, KF58333, ENMD-1198, ENMD-1237, or ganetasipib, or a salt thereof. In one embodiment, the inhibitor administered according to the provided methods interferes with HIF-dimerization. In one embodiment, the inhibitor administered according to the provided methods is acriflavine, or a salt thereof. In one embodiment, the inhibitor administered according to the provided methods is TC-S7009, PT2385, or TAT-cyclo-CLLFVY, or a salt thereof.

In a particular embodiment, the inhibitor administered according to the provided methods is ganetasipib, or a salt thereof.

In a particular embodiment, the inhibitor administered according to the provided methods is BAY 87-2243.

In some embodiments, the HIF1-α Pathway Inhibitor administered according to the provided methods is a histone deacetylase inhibitor (HDACI). In one embodiment, the administered HDACI is LW6/CAY10585 (methyl 3-(2-(4-(adamantan-1-yl)phenoxy)acetamido)-4-hydroxy-benzoate, vorinostat, romidepsin (FK228), panobinostat, belinostat, Trichostatin A (TSA), LAQ824, or phenethyl isothiocyanate, or a salt thereof.

In some embodiments, the HIF1-α Pathway Inhibitor administered according to the provided methods is a heat shock protein inhibitor. In one embodiment, the administered HIF1-α Pathway Inhibitor is an HSP90 inhibitor. In one embodiment, the administered HSP90 inhibitor is a geldanamycin or analog thereof, 17-AAG (tanespimycin: allylamino-17-demethoxy geldanamycin), 17-DMAG (alvespimycin), 17AG, radiccicol, KF58333, ENMD-1198, ENMD-1237, or ganetasipib, or a salt thereof. In a particular embodiment, the administered heat shock protein inhibitor is ganetasipib, or a salt thereof. In one embodiment, the administered HIF1-α Pathway Inhibitor is an HSP70 inhibitor. In one embodiment, the administered HSP70 inhibitor is triptolide, or a salt thereof.

In some embodiments, the HIF1-α Inhibitor administered according to the provided methods is an inhibitor of HIF transactivation. In one embodiment, the HIF1-α Inhibitor administered according to the provided methods inhibits HIF coactivator recruitment. In one embodiment, the administered HIF1-α Inhibitor is chetomin, YC-1, or KCN-1 (3,4-dimethoxy-N-[(2,2-dimethyl-2H-chromen-6-yl)methyl]-N-phenylbenzenesulfonamide), or a salt thereof. In another particular embodiment, the administered HIF1-α Inhibitor is NSC 607097, or a salt thereof. In one embodiment, the administered HIF1-α Inhibitor is a proteasome inhibitor. In a further embodiment, the administered inhibitor is bortezomib or carfilzomib, or a salt thereof. In one embodiment, the administered HIF1-α Inhibitor is indenopyrazole 21, FM19G11, flavopiridol, Amphotericin B, actinomycin, AJM290, or AW464, or a salt thereof. In one embodiment, the administered HIF1-α Inhibitor is triptolide, or a salt thereof.

In a particular embodiment, the HIF1-α Inhibitor administered according to the provided methods is YC-1, or a salt thereof.

In some embodiments, the HIF1-α Inhibitor administered according to the provided methods is an antibody that binds HIF1-α or a HIF1-α-binding antibody fragment (e.g., a single chain antibody, a single-domain antibody (e.g., the AG1-5 VHH), a Fab fragment, F(ab′)2 fragment, Fd fragment; Fv fragment, scFv, dAb fragment, or another engineered molecule, such as a diabody, triabody, tetrabody, minibody, and a minimal recognition unit). In a particular embodiment, the administered HIF1-α Inhibitor is a VHH or nanobody. In one embodiment, the administered antibody is AGI-5. In one embodiment, the administered antibody is AHPC.

In some embodiments, the HIF1-α Inhibitor administered according to the provided methods is an inhibitor of HIF1 DNA-binding. In one embodiment, the administered HIF1-α Inhibitor is echinomycin (NSC-13502) or Compound DJ12.162. In one embodiment, the administered HIF1-α Inhibitor is an anthracycline. In a further embodiment, the administered inhibitor is doxorubicin or danuorubicin. In one embodiment, the administered HIF1-α Inhibitor is a polyamide. In some embodiments, the HIF1-α Inhibitor is an antibody that binds HIF1-α or is a HIF1-α-binding antibody fragment such as a VHH or nanobody.

In some embodiments, the HIF1-A Inhibitor administered according to the provided methods is a therapeutic nucleic acid. In some embodiments the therapeutic nucleic acid is an aptamer, antisense molecule, ribozyme, a Dicer substrate, MiRNA, dsRNA, ssRNA, and shRNA). In some embodiments, the therapeutic nucleic acid is an antisense oligonucleotide.

In some embodiments, the HIF1-A Inhibitor administered according to the provided methods is an siRNA or an antisense oligonucleotide. In some embodiments, the administered HIF1-A inhibitor is RX-0047. In some embodiments, the administered HIF1-A inhibitor is EZN-2968.

Representative examples of human HIF1-A coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit HIF1-A activity can routinely be designed and prepared based on each of the above human HIF1-A transcript sequences using methods known in the art.

The administration of HIF1-A inhibitory nucleic acids or any ways of inhibiting gene expression of HIF1-A known in the art are contemplated in certain embodiments of the provided methods. Examples of inhibitory nucleic acid include but are not limited to, antisense nucleic acids such as: ENMD-1198 (small interfering RNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of HIF1-A or prevent the translation of a HIF1-A gene transcript in a cell. In some embodiments, the HIF1-A inhibitory nucleic acid administered according to the provided methods is from 16 to 1000 nucleotides in length. In certain embodiments the administered HIF1-A inhibitory nucleic acid is from 18 to 100 nucleotides long. In certain embodiments the administered HIF1-A inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the HIF1-A inhibitory nucleic acid administered according to the provided methods is capable of decreasing the expression of HIF1-A by at least 10%, 20%, 30%, or 40%, more particularly by at least 50%, 60%, or 70%, and most particularly by at least 75%, 80%, 90%, 95% or more or any range or value in between the foregoing. In some embodiments, the HIF1-A inhibitory nucleic acid administered according to the provided methods is between 17 to 25 nucleotides in length and comprises a 5′ to 3′ sequence that is at least 90% complementary to the 5′ to 3′ sequence of a mature HIF1-A mRNA (e.g., as disclosed in any one or more of GenBank accession nos. NM_001530.4, NM_181054.3, and NM_001243084.2). In some embodiments, the administered HIF1-A inhibitory nucleic acid is 17, 18, 19, 20, 21, 22, 23, 24, or 25 nucleotides in length, or any range derivable therein. In some embodiments, the administered HIF1-A inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature HIF1-A mRNA (e.g., as disclosed in any one or more of GenBank accession nos. NM_001530.4, NM_181054.3, and NM_001243084.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the HIF1-A inhibitory nucleic acid administered according to the provided methods is a miRNA mimic. In some embodiments, the administered HIF1-α Inhibitor is a miR-483 mimic.

In some embodiments, the HIF1-α Inhibitor administered according to the provided methods is a therapeutic nucleic acid. In some embodiments the therapeutic nucleic acid is an antisense oligonucleotide.

### VEGF Antagonists

In some embodiments, the disclosure provides a method of treating an ocular neovascular disease or condition in a subject in need thereof that comprises:
(a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α; and
   (i) wherein the subject is further administered an anti-VEGF Therapeutic agent, and/or
   (ii) wherein the subject has received prior treatment of an anti-VEGF Therapeutic agent.

In some embodiments, the disclosure provides a method of treating an ocular neovascular disease or condition in a subject in need thereof that comprises administering an effective amount of a VEGF antagonist in combination with a PFKFB3 inhibitor.

In some embodiments, the anti-VEGF Therapeutic agent administered to the subject is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a VEGF binding polypeptide (e.g., an Fc fusion protein), or a small molecule VEGF pathway.

In some embodiments, the anti-VEGF Therapeutic agent further administered to the subject according to the provided methods is a VEGF-Trap (see, e.g., U.S. Pat. No. 7,087,411), an anti-VEGF antibody (e.g., bevacizumab or ranibizumab), or a small molecule kinase inhibitor of VEGF receptor (e.g., sunitinib, sorafenib or pazopanib). In some embodiments, the subject is administered bevacizumab, ranibizumab, or aflibercept. In some embodiments, the subject is administered bevacizumab. In some embodiments, the subject is administered ranibizumab. In some embodiments, the subject is administered aflibercept.

In some embodiments, the anti-VEGF therapeutic agent previously administered to the subject is a VEGF-Trap (see, e.g., U.S. Pat. No. 7,087,411), anti-VEGF antibody (e.g., bevacizumab or ranibizumab), or a small molecule kinase inhibitor of VEGF receptor (e.g., sunitinib, sorafenib or pazopanib). In some embodiments, the subject was previously administered bevacizumab, ranibizumab, or aflibercept. In some embodiments, the subject was previously administered bevacizumab. In some embodiments, the subject was previously administered ranibizumab. In some embodiments, the subject was previously administered aflibercept.

### Kits for Administration of Active Agents

In another embodiment, the disclosure provides a kit containing a HIF1-α Pathway Inhibitor and a PFKFB3 inhibitor and/or other therapeutic and delivery agents. In some embodiments, a kit for preparing and/or administering a therapy described herein may be provided. The kit may comprise one or more sealed vials containing any of the pharmaceutical compositions, therapeutic agents and/or other therapeutic and delivery agents. In some embodiments, the kits comprise lipid delivery systems. In some embodiments, the lipid is in one vial, and the therapeutic agent is in a separate vial. The kit may include, for example, at least one inhibitor of PFKFB3 expression/activity, at least one inhibitor of HIF1-alpha expression/activity, and one or more reagents to prepare, formulate, and/or administer the components described herein or perform one or more steps of the methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of a therapeutic agent.

In some embodiments, kits may be provided to evaluate the expression of PFKFB3 and/or HIF-α or related molecules. Such kits can be prepared from readily available materials and reagents. For example, such kits can comprise any one or more of the following materials: enzymes, reaction tubes, buffers, detergent, primers and probes, nucleic acid amplification, and/or hybridization agents. In a particular embodiment, these kits allow a practitioner to obtain samples in blood, tears, semen, saliva, urine, tissue, serum, stool, colon, rectum, sputum, cerebrospinal fluid and supernatant from cell lysate. In another embodiment, these kits include the needed apparatus for performing RNA extraction, RT-PCR, and gel electrophoresis. Instructions for performing the assays can also be included in the kits.

Kits may comprise components, which may be individually packaged or placed in a container, such as a tube, bottle, vial, syringe, or other suitable container means. The components may include probes, primers, antibodies, arrays, negative and/or positive controls. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

The kit can further comprise reagents for labeling PFKFB3 and/or HIF-1 alpha in the sample. The kit may also include labeling reagents, including at least one of amine-modified nucleotide, poly(A) polymerase, and poly(A) polymerase buffer. Labeling reagents can include an amine-reactive dye or any dye known in the art.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits may also include a means for containing the nucleic acids, antibodies or any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. Alternatively, the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits may include a means for containing the vials in close confinement for commercial sale, such as, e.g., injection and/or blow-molded plastic containers into which the desired vials are retained. The kit may also include instructions for employing the kit components as well the use of any other reagent not included in the kit. Instructions may include variations that can be implemented.

### Methods of Treatment and Use

### Ocular Neovascular Disorders

Neovascularization within the eye contributes to visual loss in several ocular diseases, the most common of which are proliferative diabetic retinopathy, neovascular age-related macular degeneration, and retinopathy of prematurity. Together, these three diseases afflict persons in all stages of life from birth through late adulthood and account for most instances of legal blindness. In some embodiments, the disclosure provides methods and compositions for treating an ocular neovascular disorder.

In additional embodiments, the disclosure provides compositions and methods for treating an ocular neovascular disorder (OND). In one embodiment, the disclosure provides a method of treating an ocular neovascular disorder (OND) in a subject in need thereof comprising: (a) administering an effective amount of a HIF1-α Pathway Inhibitor and an PFKFB3 inhibitor to the subject; (b) administering an effective amount of a HIF1-α Pathway Inhibitor to the subject, wherein the subject has previously been administered a PFKFB3 Inhibitor; or (c) administering an effective amount of a PFKFB3 Inhibitor to the subject, wherein the subject has previously been administered a HIF1-α Pathway Inhibitor; wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor. In some embodiments, the treated ocular neovascular disease or condition is diabetic retinopathy (DR). In some embodiments, the treated ocular neovascular disease or condition is diabetic macular edema (DME). In some embodiments, the treated ocular neovascular disease or condition is age-related macular degeneration (AMD), such as wet AMD (wAMD). In some embodiments, the treated ocular neovascular disease or condition is choroidal neovascular membranes.

In some embodiments, the subject is at risk of having an OND. In some embodiments, a method provided herein (e.g., and of (a)-(c) above), is performed as a prophylactic treatment for an OND.

In some embodiments, the provided methods and compositions prevent an ocular neovascular disorder in a subject at risk for developing diabetic the ocular neovascular disorder, e.g., a subject having one or more risk factors associated with development of the ocular neovascular disorder. In some embodiments, the subject has one or more risk factors selected from hyperglycemia, hypertension, hyperlipidemia, renal disease, advanced diabetes, high average systolic blood pressure and high hemoglobin Alc.

In some embodiments, the subject has an OND. In some embodiments, the subject has been diagnosed as having an OND. Ocular neovascular disorders are often detected during a comprehensive eye exam that includes visual acuity testing (eye chart tests to measure a subjects ability to see at various distances); tonometry (measurements of pressure inside the eye); pupil dilation; and optical coherence tomography (OCT). During such exam, a physician may check for one or more of the following: changes to retinal blood vessels, including new vessel formation, swelling, and bleeding; leaking retinal blood vessels or warning signs of leaky blood vessels, such as fatty deposits, weakened vessel walls, and bulging vessel walls; swelling of the macula; changes in the lens, including changes in curvature or cataract formation; and damage to nerve tissue.

In some embodiments, the disclosure provides methods and compositions that prevent, inhibit or delay the onset of an ocular neovascular disorder by administration to a subject before the onset of the OND, e.g., before the onset of one or more symptoms thereof.

In some embodiments, the HIF1-α Pathway Inhibitor and the PFKFB3 inhibitor are administered before the onset of one or more symptoms of OND. OMDs often progresses unnoticed until vision is affected. Vision parameters impacted by OMD include overall vision, peripheral vision, night vision, color vision, distance vision, close-range vision, and vision clarity. In some embodiments, the provided compositions and methods may reduce the incidence, severity, or level of one or more of these vision parameters. In some embodiments, treating an OND according to a method provided herein comprises delaying the onset of one or more symptoms of OND.

In some embodiments, the HIF1-α Pathway Inhibitor and the PFKFB3 inhibitor are administered after the onset of one or more symptoms of an OND. In some embodiments, the provided methods and compositions can be used to treat different stages of the OND.

In some embodiments, the HIF1-α Pathway Inhibitor administered according to a method provided herein is an antibody or antigen-binding fragment thereof (e.g., a single chain antibody, a single-domain antibody ((e.g., VHH AG-1, AG-2, AG-3, AG-4, or AG-5, AHPC, or VHH212), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1.3, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., VHH AG-1, AG-2, AG-3, AG-4, or AG-5), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968, or nanobody AG-1, AG-2, AG-3, AG-4, or AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating OND is performed by co-administering the HIF1-α Pathway Inhibitor and the PFKFB3 inhibitor to the subject.

In some embodiments, the administration of the HIF1-α Pathway Inhibitor or the PFKFB3 inhibitor is an ocular administration. In some embodiments, the administration of the HIF1-α Pathway Inhibitor and the PFKFB3 inhibitor is an ocular administration. In some embodiments, the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

In some embodiments, the administration of the HIF1-α Pathway Inhibitor or the PFKFB3 inhibitor is an intravitreal administration. In some embodiments, the administration of the HIF1-α Pathway Inhibitor and the PFKFB3 inhibitor is intravitreal administration.

In some embodiments, treating an OND according to a method provided herein comprises reducing one or more symptoms of the OND in the subject compared to a control subject or compared to the subject prior to treatment with the HIF1-α Pathway Inhibitor and the PFKFB3 inhibitor. In some embodiments, the one or more reduced symptoms of the OND is selected from: retinal inflammation, acellular capillary formation, neovascularization, endothelial cell death, vascular permeability, ischemia-reperfusion injury, leakage area, and occludin disruption. In some embodiments, the one or more symptoms of OND are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the provided compositions and methods prevent or lessen the incidence, prevalence, or severity of damaged and/or leaky blood vessels in the eye.

Treatment and/or prevention of an ocular neovascular disorder can be measured by a variety of means. In some embodiments, treatment or prevention comprises treating one or more of apoptosis, inflammation, acellular capillary formation, neovascularization, retinal endothelial cell death, retinal vascular permeability, ischemic reperfusion injury, and occludin disruption in an ocular neovascular disorder in a subject in need thereof.

In some embodiments, treating OND according to a method provided herein comprises increasing one or more vision parameters in the subject compared to the vision parameters in a control subject or compared to the vision parameters of the subject prior to treatment with the HIF1-α Pathway Inhibitor and the PFKFB3 inhibitor. In some embodiments, the one or more increased vision parameters are selected from peripheral vision; night vision; color vision; distance vision; close-range vision; and vision clarity.

The disclosure provides methods of treating an ocular neovascular disorder with an anti-HIF1-alpha and/or anti-PFKFB3 antibody or antigen-binding fragment thereof. Efficacy of such treatment may be characterized, evaluated, measured, and/or monitored based on several parameters.

In some embodiments, the provided methods result in decreased apoptosis and/or endothelial cell death within the eye. Cell death may be monitored according to known methods. Illustrative methods for detecting cell death include but are not limited to, nuclear staining techniques such as propidium iodide, Hoechst-33342, 4′, 6-diamidino-2-phenylindole (DAPI), and Acridine orange-Ethidium bromide staining. Nonnuclear staining techniques include but are not limited to, Annexin-V staining.

In some embodiments, the provided methods prevent ischemia-reperfusion (IR) injury. Methods for detecting IR injury are known in the art and include fluorescein analysis, fluorescent zinc 2,2′-dipicolylamine coordination complex PSVue.RTM.794, 99mTc glucarate, and electroretinography.

In some embodiments, the provided methods reduce levels of inflammatory cytokines, such as TNFα, IL-1β, IL-6, or MCP1. Cytokine levels may be monitored via enzyme-linked immunosorbant assay (ELISA), Luminex, Cytokine Bead Array, Proteo Plex, FAST Quant, and the like.

In some embodiments, the provided methods reduce ocular vascular permeability, ocular neovascularization, or other symptoms of ocular health. In some embodiments, the provided methods may prevent typical symptoms of an ocular neovascular disorder in the ocular vasculature or may prevent further deterioration. Vascular permeability and other measures of ocular vascular health may be measured by, e.g., fluorescein angiography.

In some embodiments, the provided methods improve one or more vision parameters or prevent the decline of one or more vision parameters. Vision parameters include: poor night vision, blurred vision, floating spots, black spots or flashing lights in the vision field, fluctuating vision, impaired color vision, dark or empty areas in your vision, vision loss, and sudden severe painless vision loss. In some embodiments, subjects receiving treatment according to the provided methods may experience one or more of the following effects: improved vision, reduced vision loss, improved night vision, improved low light vision, improved reading ability, improved peripheral vision, reduced spots in the vision field, reduced flashing lights in the vision field, reduced pain, and improved eye appearance. Many of these parameters may be monitored through routine eye examination.

In some embodiments, the provided methods prevent, reduce or delay the OND. The methods may be administered to patients at risk for developing the OND. In such subjects, prevention of an ocular neovascular disorder may be monitored by maintenance of vision or by lack of typical hallmarks of the OND. For example, subjects to whom an effective amount of a HIF1-alpha inhibitor and PFKFB3 inhibitor is administered prophylactically may not experience or may experience a reduced incidence of one or more of the following symptoms: microaneurysms, hemorrhages, intraretinal microvascular abnormalities, venous beading, cotton wool spots, the formation of new blood vessels (neovascularization) elsewhere and on the optic nerve, fibrous proliferation elsewhere and on the optic nerve, preretinal and vitreous hemorrhage, retinal detachment due to scar tissue formation, glaucoma, poor night vision, blurred vision, floating spots, black spots or flashing lights in the vision field, fluctuating vision, impaired color vision, dark or empty areas in your vision, vision loss, sudden severe painless vision loss, traction retinal detachment, macular edema, venous dilation, and intraretinal microvascular abnormalities.

In some embodiments, the provided methods of treating an ocular neovascular disorder affect one or more parameters of the retinal vasculature, including, but not limited to: permeability, NFkB levels, inflammatory marker levels, apoptosis incidence, sphingolipid metabolism, and reendothelialization. In some embodiments, a method of treating an ocular neovascular disorder disclosed herein decreases retinal vascular permeability. Retinal vascular permeability may be monitored via fluorescence, tracer dyes, optical exam, and the like. In some embodiments, a method of treating an ocular neovascular disorder disclosed herein decreases NFkB and/or other inflammatory marker levels in the retinal vasculature. As discussed above, inflammatory cytokine levels may be measured through conventional means (e.g., ELISA). In some embodiments, a method of treating an ocular neovascular disorder disclosed herein decreases the incidence of apoptosis in the retinal vasculature. As disclosed above, apoptosis may be measured using conventional means, e.g., nuclear and nonnuclear staining techniques.

In additional embodiments, the provided methods include further administering an anti-VEGF therapeutic agent to the subject. In some embodiments, the anti-VEGF therapeutic agent is bevacizumab, ranibizumab, or aflibercept. In one embodiment, the anti-VEGF therapeutic agent is bevacizumab. In one embodiment, the anti-VEGF therapeutic agent is ranibizumab. In one embodiment, the anti-VEGF therapeutic agent is aflibercept.

In some embodiments, the subject receiving a treatment provided herein has received a prior treatment for OND. In some embodiments, the prior treatment is a therapeutic procedure selected from a vitrectomy and laser surgery, or a therapeutic agent selected from a steroid and an anti-vascular endothelial growth factor (VEGF) therapy. In a particular embodiment, the prior treatment is an anti-vascular endothelial growth factor (VEGF) therapy. In some embodiments, the prior anti-VEGF therapy included bevacizumab, ranibizumab, or aflibercept. In one embodiment, the prior anti-VEGF therapy included bevacizumab. In one embodiment, the prior anti-VEGF therapy included ranibizumab. In one embodiment, the prior anti-VEGF therapy included aflibercept. In some embodiments, the subject failed to respond to the prior treatment for OND. In such embodiments, a “failure to respond” indicates that the previous treatment failed to ameliorate and/or improve one or more symptoms of diabetic retinopathy. In such embodiments, a “failure to respond” indicates that the previous treatment failed to ameliorate and/or improve one or more symptoms of diabetic retinopathy. In some embodiments, the previous therapy may have failed to produce an improvement in vision or in retinal vascular health. In some embodiments, the previous therapy may have shown some results, but may not have achieved the desired performance or may have stopped showing efficacy after some period of time. In some embodiments, the subject may have partially responded to a previous ocular neovascular disorder treatment: i.e., one or more symptoms of an ocular neovascular disorder were not sufficiently ameliorated and/or the effects of the previous treatment were not sufficiently durable.

Existing treatments for ocular neovascular disorders include anti-VEGF therapy, steroids, laser surgery, and vitrectomy.

### Diabetic Retinopathy

In some embodiments, the disclosure provides methods and compositions for treating diabetic retinopathy. Diabetic retinopathy refers to a medical condition in which damage occurs to the retina due to diabetes mellitus. Chronically high blood sugar from diabetes is associated with damage to the tiny blood vessels in the retina, leading to diabetic retinopathy. Diabetic retinopathy can cause blood vessels in the retina to leak fluid or hemorrhage, distorting vision. In its most advanced stage, new abnormal blood vessels proliferate on the surface of the retina, which can lead to scarring and cell loss in the retina.

In additional embodiments, the disclosure provides compositions and methods for treating diabetic retinopathy (DR). In one embodiment, the disclosure provides a method of treating diabetic retinopathy (DR) in a subject in need thereof comprising: (a) administering an effective amount of a HIF1-α Pathway Inhibitor and an PFKFB3 inhibitor to the subject; (b) administering an effective amount of a HIF1-α Pathway Inhibitor to the subject, wherein the subject has previously been administered a PFKFB3 Inhibitor; or (c) administering an effective amount of a PFKFB3 Inhibitor to the subject, wherein the subject has previously been administered a HIF1-α Pathway Inhibitor; wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having DR. In some embodiments, a method provided herein (e.g., and of (a)-(c) above), is performed as a prophylactic treatment for DR.

In some embodiments, the provided methods and compositions prevent diabetic retinopathy in a subject at risk for developing diabetic retinopathy, e.g., a subject having one or more risk factors associated with development of diabetic retinopathy. In some embodiments, the subject has one or more risk factors selected from hyperglycemia, hypertension, hyperlipidemia, renal disease, and advanced diabetes (e.g., indicated by use of insulin and oral diabetes treatments versus pills alone or use of pills alone versus no treatment). In some embodiments, the subject has one or more risk factors selected from high average systolic blood pressure and high hemoglobin Alc.

In some embodiments, the subject has DR. In some embodiments, the subject has been diagnosed as having DR. Diabetic retinopathy and diabetic macular edema may be detected during a comprehensive eye exam that includes visual acuity testing (eye chart tests to measure a subjects ability to see at various distances); tonometry (measurements of pressure inside the eye); pupil dilation; and optical coherence tomography (OCT). During such exam, a physician may check for one or more of the following: changes to retinal blood vessels, including new vessel formation, swelling, and bleeding; leaking retinal blood vessels or warning signs of leaky blood vessels, such as fatty deposits, weakened vessel walls, and bulging vessel walls; swelling of the macula; changes in the lens, including changes in curvature or cataract formation; and damage to nerve tissue.

In some embodiments, the disclosure provides methods and compositions that prevent, inhibit or delay the onset of diabetic retinopathy diabetic retinopathy by administration to a diabetic subject before the onset of diabetic retinopathy, e.g., before the onset of one or more symptoms thereof. Among the most consistent risk factors, duration of diabetes is a strong predictor for development and progression of the retinopathy. Among subjects with younger-onset diabetes, the prevalence is estimated at approximately 8% at 3 years, 25% at 5 years, 60% at 10 years, and 80% at 15 years. In some embodiments, the provided methods and compositions maybe used to delay the onset of diabetic retinopathy, e.g., to more than 3 years, more than 5 years, more than 10 years, or more than 15 years after development of diabetes.

In some embodiments, the HIF1-α Pathway Inhibitor and the PFKFB3 inhibitor are administered before the onset of one or more symptoms of DR. The disease often progresses unnoticed until it affects vision. Bleeding from retinal blood vessels during the early stages of disease can cause the appearance of “floating” spots or dark strings, which may clear on their own. Without prompt treatment, bleeding often recurs, increasing the risk of permanent vision loss. If diabetic macular edema occurs, it can cause blurred vision. In some embodiments, the provided compositions and methods may reduce the incidence, severity, or level of floating spots, retinal bleeding, vision loss, and/or blurred vision. In some embodiments, one or more parameters of vision may be improved by the provided methods, including, but not limited to, overall vision, peripheral vision, night vision, color vision, distance vision, close-range vision, and vision clarity.

In some embodiments, treating DR according to a method provided herein comprises delaying the onset of one or more symptoms of DR.

In some embodiments, the HIF1-α Pathway Inhibitor and the PFKFB3 inhibitor are administered after the onset of one or more symptoms of DR. In some embodiments, the provided methods and compositions can be used to treat different stages of diabetic retinopathy. Diabetic retinopathy may progress through a non-proliferative stage, also referred to as early stage, and a proliferative stage, also referred to as late stage.

In some embodiments, the disclosure provides methods and compositions that treat diabetic retinopathy during the non-proliferative stage. Non-proliferative diabetic retinopathy (NPDR) may be mild, moderate, or severe. Mild NPDR is characterized by microaneurysms in the retina's blood vessels. These microaneurysms may leak fluid into the retina. As the disease progresses to the moderate NPDR stage, retinal blood vessels can become distorted and lose their ability to transport blood, resulting in characteristic changes to the appearance of the retina and may contribute to diabetic macular edema. At the severe NPDR stage, many more blood vessels are blocked, depriving blood supply to areas of the retina. These areas then secrete pro-angiogenic growth factors. Angiogenic molecules in the eye include VEGF, FGF, P1GF, TGF-alpha, TGF-beta, IGF, PDGF, MMPs, HGF/SF, TNF-alpha, CTGF, IL-1, IL-8, MCP-1, leptin, integrins, and angiogenin. In some embodiments, the provided methods are characterized by preventing or decreasing one or more markers of diabetic retinopathy. In some embodiments, the provided methods may prevent, reduce, inhibit, or lower the level of retinal microaneurysms, retinal fluid leakage, diabetic macular edema, and/or retinal pro-angiogenic growth factors. In some embodiments, one or more of VEGF, FGF, P1GF, TGF-alpha, TGF-beta, IGF, PDGF, MMPs, HGF/SF, TNF-alpha, CTGF, IL-1, IL-8, MCP-1, integrins, and angiogenin are decreased by the provided methods.

In some embodiments, the disclosure provides methods and compositions that treat diabetic retinopathy during the proliferative stage. Proliferative diabetic retinopathy (PDR) is the advanced stage of disease. At this stage, pro-angiogenic growth factors secreted by the retina trigger the proliferation of new blood vessels, which grow along the inside surface of the retina and into the vitreous gel. The new blood vessels are fragile and more likely to leak and bleed. Accompanying scar tissue can contract and cause retinal detachment which can lead to permanent vision loss. In some embodiments, the provided methods may prevent, reduce, inhibit, or lower the level or severity of retinal neovascularization, retinal hemorrhage, retinal scarring, retinal detachment, and/or vision loss.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1.3, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968, nanobody AG-1-5 or nanobody AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof. In some embodiments, a method provided herein for treating DR is performed by co-administering the HIF1-α Pathway Inhibitor and the PFKFB3 inhibitor to the subject.

In some embodiments, the administration of the HIF1-α Pathway Inhibitor or the PFKFB3 inhibitor is an ocular administration. In some embodiments, the administration of the HIF1-α Pathway Inhibitor and the PFKFB3 inhibitor is an ocular administration. In some embodiments, the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

In some embodiments, the administration of the HIF1-α Pathway Inhibitor or the PFKFB3 inhibitor is an intravitreal administration. In some embodiments, the administration of the HIF1-α Pathway Inhibitor and the PFKFB3 inhibitor is intravitreal administration.

In some embodiments, treating DR according to a method provided herein comprises reducing one or more symptoms of DR in the subject compared to a control subject or compared to the subject prior to treatment with the HIF1-α Pathway Inhibitor and the PFKFB3 inhibitor. In some embodiments, the one or more reduced symptoms of DR is selected from: retinal inflammation, acellular capillary formation, retinal neovascularization, retinal endothelial cell death, retinal vascular permeability, retinal ischemia-reperfusion injury, retinal leakage area, and occludin disruption. In some embodiments, the one or more symptoms of DR are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the provided compositions and methods prevent or lessen the incidence, prevalence, or severity of damaged and/or leaky blood vessels in the eye.

Treatment and/or prevention of diabetic retinopathy can be measured by a variety of means. In some embodiments, treatment or prevention comprises treating one or more of apoptosis, inflammation, acellular capillary formation, neovascularization, retinal endothelial cell death, retinal vascular permeability, ischemic reperfusion injury, and occludin disruption in diabetic retinopathy in a subject in need thereof.

In some embodiments, treating DR according to a method provided herein comprises increasing one or more vision parameters in the subject compared to the vision parameters in a control subject or compared to the vision parameters of the subject prior to treatment with the HIF1-α Pathway Inhibitor and the PFKFB3 inhibitor. In some embodiments, the one or more increased vision parameters are selected from peripheral vision; night vision; color vision; distance vision; close-range vision; and vision clarity.

The disclosure provides methods of treating diabetic retinopathy with an anti-HIF1-alpha and/or anti-PFKFB3 antibody or antigen-binding fragment thereof. Efficacy of such treatment may be characterized, evaluated, measured, and/or monitored based on several parameters.

In some embodiments, the provided methods result in decreased apoptosis and/or endothelial cell death within the eye. Cell death may be monitored according to known methods. Illustrative methods for detecting cell death include but are not limited to, nuclear staining techniques such as propidium iodide, Hoechst-33342, 4′, 6-diamidino-2-phenylindole (DAPI), and Acridine orange-Ethidium bromide staining. Nonnuclear staining techniques include but are not limited to, Annexin-V staining.

In some embodiments, the provided methods prevent ischemia-reperfusion (IR) injury. Methods for detecting IR injury are known in the art and include fluorescein analysis, fluorescent zinc 2,2′-dipicolylamine coordination complex PSVue.RTM.794, 99mTc glucarate, and electroretinography.

In some embodiments, the provided methods reduce levels of inflammatory cytokines, such as TNFα, IL-1β, IL-6, or MCP1. Cytokine levels may be monitored via enzyme-linked immunosorbant assay (ELISA), Luminex, Cytokine Bead Array, Proteo Plex, FAST Quant, and the like.

In some embodiments, the provided methods reduce retinal vascular permeability, retinal neovascularization, or other symptoms of retinal health. In some embodiments, the provided methods may prevent typical symptoms of diabetic retinopathy in the retinal vasculature or may prevent further deterioration. Vascular permeability and other measures of retinal vascular health may be measured by, e.g., fluorescein angiography.

In some embodiments, the provided methods improve one or more vision parameters or prevent the decline of one or more vision parameters. Vision parameters include: poor night vision, blurred vision, floating spots, black spots or flashing lights in the vision field, fluctuating vision, impaired color vision, dark or empty areas in your vision, vision loss, sudden severe painless vision loss. In some embodiments, subjects receiving treatment according to the provided methods may experience one or more of the following effects: improved vision, reduced vision loss, improved night vision, improved low light vision, improved reading ability, improved peripheral vision, reduced spots in the vision field, reduced flashing lights in the vision field, reduced pain, and improved eye appearance. Many of these parameters may be monitored through routine eye examination.

In some embodiments, the provided methods prevent diabetic retinopathy. The methods may be administered to patients at risk for developing diabetic retinopathy. In such subjects, prevention of diabetic retinopathy may be monitored by maintenance of vision or by lack of typical hallmarks of diabetic retinopathy. For example, subjects to whom an effective amount of a HIF1-alpha inhibitor and PFKFB3 inhibitor is administered prophylactically may not experience or may experience a reduced incidence of one or more of the following symptoms: microaneurysms, hemorrhages, intraretinal microvascular abnormalities, venous beading, cotton wool spots, the formation of new blood vessels (neovascularization) elsewhere and on the optic nerve, fibrous proliferation elsewhere and on the optic nerve, preretinal and vitreous hemorrhage, retinal detachment due to scar tissue formation, glaucoma, poor night vision, blurred vision, floating spots, black spots or flashing lights in the vision field, fluctuating vision, impaired color vision, dark or empty areas in your vision, vision loss, sudden severe painless vision loss, traction retinal detachment, macular edema, venous dilation, and intraretinal microvascular abnormalities.

In some embodiments, the provided methods prevent or reduce macular edema. Macular edema may be seen on slit-lamp biomicroscopy as elevation and blurring of retinal layers.

In some embodiments, the provided methods delay the onset of diabetic retinopathy. Accordingly, the provided methods delay the average onset of diabetic retinopathy to greater than 5 years, greater than 10 years, greater than 11 years, greater than 12 years, greater than 13 years, greater than 14 years, greater than 15 years, greater than 16 years, greater than 17 years, greater than 18 years, greater than 19 years, or greater than 20 years after initial diabetes diagnosis.

In some embodiments, the provided methods reduce, ameliorate, lessen the severity of, or reverse one or more symptoms of diabetic retinopathy. In some embodiments, methods of treating diabetic retinopathy with anti-HIF1-alpha and/or anti-PFKFB3 antibodies or antigen-binding fragments thereof may reduce, ameliorate, lessen the severity of, or reverse one or more of the following symptoms: microaneurysms, hemorrhages, intraretinal microvascular abnormalities, venous beading, cotton wool spots, the formation of new blood vessels (neovascularization) elsewhere and on the optic nerve, fibrous proliferation elsewhere and on the optic nerve, preretinal and vitreous hemorrhage, retinal detachment due to scar tissue formation, loss of vision, glaucoma, poor night vision, blurred vision, floating spots, black spots or flashing lights in the vision field, sudden severe painless vision loss, traction retinal detachment, macular edema, venous dilation, and intraretinal microvascular abnormalities.

In some embodiments, the provided methods of treating diabetic retinopathy affect one or more parameters of the retinal vasculature, including, but not limited to: permeability, NFkB levels, inflammatory marker levels, apoptosis incidence, sphingolipid metabolism, and reendothelialization. In some embodiments, a method of treating diabetic retinopathy disclosed herein decreases retinal vascular permeability. Retinal vascular permeability may be monitored via fluorescence, tracer dyes, optical exam, and the like. In some embodiments, a method of treating diabetic retinopathy disclosed herein decreases NFkB and/or other inflammatory marker levels in the retinal vasculature. As discussed above, inflammatory cytokine levels may be measured through conventional means (e.g., ELISA). In some embodiments, a method of treating diabetic retinopathy disclosed herein decreases the incidence of apoptosis in the retinal vasculature. As disclosed above, apoptosis may be measured using conventional means, e.g., nuclear and nonnuclear staining techniques.

In additional embodiments, the provided methods include further administering an anti-VEGF therapeutic agent to the subject. In some embodiments, the anti-VEGF therapeutic agent is bevacizumab, ranibizumab, or aflibercept. In one embodiment, the anti-VEGF therapeutic agent is bevacizumab. In one embodiment, the anti-VEGF therapeutic agent is ranibizumab. In one embodiment, the anti-VEGF therapeutic agent is aflibercept.

In some embodiments, the subject receiving a treatment provided herein has received a prior treatment for DR. In some embodiments, the prior treatment is a therapeutic procedure selected from a vitrectomy and laser surgery, or a therapeutic agent selected from a steroid and an anti-vascular endothelial growth factor (VEGF) therapy. In a particular embodiment, the prior treatment is an anti-vascular endothelial growth factor (VEGF) therapy. In some embodiments, the prior anti-VEGF therapy included bevacizumab, ranibizumab, or aflibercept. In one embodiment, the prior anti-VEGF therapy included bevacizumab. In one embodiment, the prior anti-VEGF therapy included ranibizumab. In one embodiment, the prior anti-VEGF therapy included aflibercept. In some embodiments, the subject failed to respond to the prior treatment for DR. In such embodiments, a “failure to respond” indicates that the previous treatment failed to ameliorate and/or improve one or more symptoms of diabetic retinopathy. In such embodiments, a “failure to respond” indicates that the previous treatment failed to ameliorate and/or improve one or more symptoms of diabetic retinopathy. In some embodiments, the previous therapy may have failed to produce an improvement in vision or in retinal vascular health. In some embodiments, the previous therapy may have shown some results, but may not have achieved the desired performance or may have stopped showing efficacy after some period of time. In some embodiments, the subject may have partially responded to a previous diabetic retinopathy treatment: i.e., one or more symptoms of diabetic retinopathy were not sufficiently ameliorated and/or the effects of the previous treatment were not sufficiently durable.

Existing treatments for diabetic retinopathy include anti-VEGF therapy, steroids, laser surgery, and vitrectomy.

### Diabetic Macular Edema

Diabetic maculopathy such as diabetic macular edema (DME) together with diabetic macular edema, is considered as one of the important retinal diseases in patients with diabetes mellitus. Macular edema caused by the rupture of the hematorretinal barrier of the retina in a retinal vascular endothelial cell or a retinal pigment epithelial cell accounts for approximately 90% of maculopathies and is a leading cause of visual acuity deterioration in maculopathy This deterioration of visual acuity does not cause blindness although it causes extreme deterioration of visual acuity called social blindness, which makes everyday life difficult.

In some embodiments, the disclosure provides methods and compositions for treating diabetic macular edema. Diabetic macular edema refers to a medical condition in which damage occurs to the retina due to diabetes mellitus. Chronically high blood sugar from diabetes is associated with damage to the tiny blood vessels in the retina, leading to diabetic macular edema. Diabetic macular edema can cause blood vessels in the retina to leak fluid or hemorrhage, distorting vision. In its most advanced stage, new abnormal blood vessels proliferate on the surface of the retina, which can lead to scarring and cell loss in the retina.

In additional embodiments, the disclosure provides compositions and methods for treating diabetic macular edema (DME). In one embodiment, that disclosure provides a method of treating diabetic macular edema (DME) in a subject in need thereof comprising: (a) administering an effective amount of a HIF1-α Pathway Inhibitor and an PFKFB3 inhibitor to the subject; (b) administering an effective amount of a HIF1-α Pathway Inhibitor to the subject, wherein the subject has previously been administered a PFKFB3 Inhibitor; or (c) administering an effective amount of a PFKFB3 Inhibitor to the subject, wherein the subject has previously been administered a HIF1-α Pathway Inhibitor; wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having DME. In some embodiments, a method provided herein (e.g., and of (a)-(c) above), is performed as a prophylactic treatment for DME. In some embodiments, the subject has diabetes mellitus. In some embodiments, the subject has diabetic retinopathy.

In some embodiments, the provided methods and compositions prevent diabetic macular edema in a subject at risk for developing diabetic macular edema, e.g., a subject having one or more risk factors associated with development of diabetic macular edema. In some embodiments, the subject has one or more risk factors selected from hyperglycemia, hypertension, hyperlipidemia, renal disease, and advanced diabetes (e.g., indicated by use of insulin and oral diabetes treatments versus pills alone or use of pills alone versus no treatment).

In some embodiments, the disclosure provides methods and compositions that prevent, inhibit or delay the onset of diabetic macular edema diabetic by administration to a diabetic subject before the onset of diabetic macular edema, e.g., before the onset of one or more symptoms thereof.

In some embodiments, the HIF1-α Pathway Inhibitor and the PFKFB3 inhibitor are administered before the onset of one or more symptoms of DME. In some embodiments, the provided compositions and methods may reduce the incidence, severity, or level of floating spots, retinal bleeding, vision loss, and/or blurred vision. In some embodiments, one or more parameters of vision may be improved by the provided methods, including, but not limited to, overall vision, peripheral vision, night vision, color vision, distance vision, close-range vision, and vision clarity.

In some embodiments, treating DME according to a method provided herein comprises delaying the onset of one or more symptoms of DME.

In some embodiments, the HIF1-α Pathway Inhibitor and the PFKFB3 inhibitor are administered after the onset of one or more symptoms of DME. In some embodiments, the disclosure provides methods and compositions that treat local macular edema.

In some embodiments, the disclosure provides methods and compositions that treat diffuse macular edema.

In some embodiments, the HIF1-α Pathway Inhibitor administered according to a method provided herein is an antibody or antigen-binding fragment thereof (e.g., a single chain antibody, a single-domain antibody (e.g., VHHAG-1, AG-2, AG-3, AG-4, or AG-5, or VHH212), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1.3, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968, nanobody AG-1-5 or nanobody AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating DME is performed by co-administering the HIF1-α Pathway Inhibitor and the PFKFB3 inhibitor to the subject.

In some embodiments, the administration of the HIF1-α Pathway Inhibitor or the PFKFB3 inhibitor is an ocular administration. In some embodiments, the administration of the HIF1-α Pathway Inhibitor and the PFKFB3 inhibitor is an ocular administration. In some embodiments, the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

In some embodiments, the administration of the HIF1-α Pathway Inhibitor or the PFKFB3 inhibitor is an intravitreal administration. In some embodiments, the administration of the HIF1-α Pathway Inhibitor and the PFKFB3 inhibitor is intravitreal administration.

In some embodiments, treating DME according to a method provided herein comprises reducing one or more symptoms of DME in the subject compared to a control subject or compared to the subject prior to treatment with the HIF1-α Pathway Inhibitor and the PFKFB3 inhibitor. In some embodiments, the one or more reduced symptoms of DME is selected from: retinal inflammation, acellular capillary formation, retinal neovascularization, retinal endothelial cell death, retinal vascular permeability, retinal ischemia-reperfusion injury, retinal leakage area, and occludin disruption. In some embodiments, the one or more symptoms of DME are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the provided compositions and methods prevent or lessen the incidence, prevalence, or severity of damaged and/or leaky blood vessels in the eye.

Treatment and/or prevention of diabetic macular edema can be measured by a variety of means. In some embodiments, treatment or prevention comprises treating one or more of apoptosis, inflammation, acellular capillary formation, neovascularization, retinal endothelial cell death, retinal vascular permeability, ischemic reperfusion injury, and occludin disruption in diabetic macular edema in a subject in need thereof.

In some embodiments, treating DME according to a method provided herein comprises increasing one or more vision parameters in the subject compared to the vision parameters in a control subject or compared to the vision parameters of the subject prior to treatment with the HIF1-α Pathway Inhibitor and the PFKFB3 inhibitor. In some embodiments, the one or more increased vision parameters are selected from blurred vision, visual distortion, and speckles in the visual field (sometimes referred to as “fly mosquito disease”).

The disclosure provides methods of treating diabetic macular edema with an anti-HIF1-alpha and/or anti-PFKFB3 antibody or antigen-binding fragment thereof. Efficacy of such treatment may be characterized, evaluated, measured, and/or monitored based on several parameters.

In some embodiments, the provided methods result in decreased apoptosis and/or endothelial cell death within the eye. Cell death may be monitored according to known methods. Illustrative methods for detecting cell death include but are not limited to, nuclear staining techniques such as propidium iodide, Hoechst-33342, 4′, 6-diamidino-2-phenylindole (DAPI), and Acridine orange-Ethidium bromide staining. Nonnuclear staining techniques include but are not limited to, Annexin-V staining.

In some embodiments, the provided methods prevent ischemia-reperfusion (IR) injury. Methods for detecting IR injury are known in the art and include fluorescein analysis, fluorescent zinc 2,2′-dipicolylamine coordination complex PSVue.RTM.794, 99mTc glucarate, and electroretinography.

In some embodiments, the provided methods reduce levels of inflammatory cytokines, such as TNFα, IL-1β, IL-6, or MCP1. Cytokine levels may be monitored via enzyme-linked immunosorbant assay (ELISA), Luminex, Cytokine Bead Array, Proteo Plex, FAST Quant, and the like.

In some embodiments, the provided methods reduce retinal vascular permeability, retinal neovascularization, or other symptoms of retinal health. In some embodiments, the provided methods may prevent typical symptoms of diabetic macular edema in the retinal vasculature or may prevent further deterioration. Vascular permeability and other measures of retinal vascular health may be measured by, e.g., fluorescein angiography.

In some embodiments, the provided methods improve one or more vision parameters or prevent the decline of one or more vision parameters. Vision parameters include: blurred vision, visual distortion, and speckles in the visual field. In some embodiments, subjects receiving treatment according to the provided methods may experience one or more of the following effects: improved vision, reduced vision loss, improved clarity and reduced speckles in the visual field. Many of these parameters may be monitored through routine eye examination.

In some embodiments, the provided methods prevent or reduce macular edema. Macular edema may be seen on slit-lamp biomicroscopy as elevation and blurring of retinal layers.

In some embodiments, the provided methods delay the onset of diabetic macular edema. Accordingly, the provided methods delay the average onset of diabetic macular edema to greater than 5 years, greater than 10 years, greater than 11 years, greater than 12 years, greater than 13 years, greater than 14 years, greater than 15 years, greater than 16 years, greater than 17 years, greater than 18 years, greater than 19 years, or greater than 20 years after initial diabetes diagnosis.

In some embodiments, the provided methods reduce, ameliorate, lessen the severity of, or reverse one or more symptoms of diabetic macular edema. In some embodiments, methods of treating diabetic macular edema with anti-HIF1-alpha and/or anti-PFKFB3 antibodies or antigen-binding fragments thereof may reduce, ameliorate, lessen the severity of, or reverse one or more of the following symptoms: microaneurysms, hemorrhages, intraretinal microvascular abnormalities, the formation of new blood vessels (neovascularization) impaired vision, macular edema, and intraretinal microvascular abnormalities.

In some embodiments, the provided methods of treating diabetic macular edema affect one or more parameters of the retinal vasculature, including, but not limited to: permeability, NFkB levels, inflammatory marker levels, apoptosis incidence, sphingolipid metabolism, and reendothelialization. In some embodiments, a method of treating diabetic macular edema disclosed herein decreases retinal vascular permeability. Retinal vascular permeability may be monitored via fluorescence, tracer dyes, optical exam, and the like. In some embodiments, a method of treating diabetic macular edema disclosed herein decreases NFkB and/or other inflammatory marker levels in the retinal vasculature. As discussed above, inflammatory cytokine levels may be measured through conventional means (e.g., ELISA). In some embodiments, a method of treating diabetic macular edema disclosed herein decreases the incidence of apoptosis in the retinal vasculature. As disclosed above, apoptosis may be measured using conventional means, e.g., nuclear and nonnuclear staining techniques.

In additional embodiments, the provided methods include further administering an anti-VEGF therapeutic agent to the subject. In some embodiments, the anti-VEGF therapeutic agent is bevacizumab, ranibizumab, or aflibercept. In one embodiment, the anti-VEGF therapeutic agent is bevacizumab. In one embodiment, the anti-VEGF therapeutic agent is ranibizumab. In one embodiment, the anti-VEGF therapeutic agent is aflibercept.

In some embodiments, the subject receiving a treatment provided herein has received a prior treatment for DME. In some embodiments, the prior treatment is a therapeutic procedure selected from a vitrectomy and laser surgery, or a therapeutic agent selected from a steroid and an anti-vascular endothelial growth factor (VEGF) therapy. In a particular embodiment, the prior treatment is an anti-vascular endothelial growth factor (VEGF) therapy. In some embodiments, the prior anti-VEGF therapy included bevacizumab, ranibizumab, or aflibercept. In one embodiment, the prior anti-VEGF therapy included bevacizumab. In one embodiment, the prior anti-VEGF therapy included ranibizumab. In one embodiment, the prior anti-VEGF therapy included aflibercept. In some embodiments, the subject failed to respond to the prior treatment for DME. In such embodiments, a “failure to respond” indicates that the previous treatment failed to ameliorate and/or improve one or more symptoms of diabetic macular edema. In such embodiments, a “failure to respond” indicates that the previous treatment failed to ameliorate and/or improve one or more symptoms of diabetic macular edema. In some embodiments, the previous therapy may have failed to produce an improvement in vision or in retinal vascular health. In some embodiments, the previous therapy may have shown some results, but may not have achieved the desired performance or may have stopped showing efficacy after some period of time. In some embodiments, the subject may have partially responded to a previous diabetic macular edema treatment: i.e., one or more symptoms of diabetic macular edema were not sufficiently ameliorated and/or the effects of the previous treatment were not sufficiently durable.

Existing treatments for diabetic macular edema include anti-VEGF therapy and laser surgery.

### Age-Related Macular Degeneration

In some embodiments, the disclosure provides methods and compositions for treating age-related macular degeneration.

The central part of the retina, called the macula, is responsible for vision that is needed for reading and other detailed work. Damage to the macula results in poor vision. The most common disease process that affects the macula is AMD. In patients with AMD, retinal photoreceptor and pigment epithelial cells in the macula die over the course of several years. The cell death and gradual visual loss usually do not begin until age 60 or older, hence the name age-related macular degeneration.

There are two types of AMD: dry macular degeneration and wet macular degeneration. Dry macular degeneration, although more common, typically results in a less severe, more gradual loss of vision Patients with wet macular degeneration develop new blood vessels under the retina. Patients with wet macular degeneration develop new blood vessels under the retina. As the photoreceptor and RPE cells slowly degenerate, there is a tendency for blood vessels to grow from their normal location in the choroid into an abnormal location beneath the retina. This abnormal new blood vessel growth is called choroidal neovascularization (CNV). The abnormal blood vessels leak and bleed, causing hemorrhage, swelling, scar tissue, and severe loss of central vision. Only 10% of patients with AMD have the wet type, but it is responsible for 90% of all blindness resulting from AMD.

In some embodiments, the disclosure provides compositions and methods for treating age-related macular degeneration (AMD). In some embodiments, the disclosure provides compositions and methods for treating wet AMD (wAMD). In some embodiments, the disclosure provides compositions and methods for treating dry AMD (dAMD). In one embodiment, that disclosure provides a method of treating AMD (e.g., wAMD or dAMD) in a subject in need thereof comprising: (a) administering an effective amount of a HIF1-α Pathway Inhibitor and an PFKFB3 inhibitor to the subject; (b) administering an effective amount of a HIF1-α Pathway Inhibitor to the subject, wherein the subject has previously been administered a PFKFB3 Inhibitor; or (c) administering an effective amount of a PFKFB3 Inhibitor to the subject, wherein the subject has previously been administered a HIF1-α Pathway Inhibitor; wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having AMD. In some embodiments the subject is at risk of having wAMD. In some embodiments the subject is at risk of having dAMD. In some embodiments, a method provided herein (e.g., and of (a)-(c) above), is performed as a prophylactic treatment for AMD.

In some embodiments, the provided methods and compositions prevent age-related macular degeneration in a subject at risk for developing age-related macular degeneration, e.g., a subject having one or more risk factors associated with development of age-related macular degeneration.

In some embodiments, the HIF1-α Pathway Inhibitor and the PFKFB3 inhibitor are administered before the onset of one or more symptoms of AMD. In some embodiments, the provided compositions and methods may reduce the incidence, severity, or level of choroidal neovascularization, ocular bleeding, hemorrhage or loss of central vision.

In some embodiments, treating AMD (e.g., wAMD) according to a method provided herein comprises delaying the onset of one or more symptoms of AMD.

In some embodiments, the HIF1-α Pathway Inhibitor and the PFKFB3 inhibitor are administered after the onset of one or more symptoms of AMD. In some embodiments, the provided methods and compositions can be used to treat different stages of age-related macular degeneration.

In some embodiments, the disclosure provides methods and compositions that treat wet AMD.

In some embodiments, the disclosure provides methods and compositions that treat dry AMD.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1.3, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968, nanobody AG-1-5 or nanobody AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating AMD (e.g., wAMD) is performed by co-administering the HIF1-α Pathway Inhibitor and the PFKFB3 inhibitor to the subject.

In some embodiments, the administration of the HIF1-α Pathway Inhibitor or the PFKFB3 inhibitor is an ocular administration. In some embodiments, the administration of the HIF1-α Pathway Inhibitor and the PFKFB3 inhibitor is an ocular administration. In some embodiments, the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

In some embodiments, the administration of the HIF1-α Pathway Inhibitor or the PFKFB3 inhibitor is an intravitreal administration. In some embodiments, the administration of the HIF1-α Pathway Inhibitor and the PFKFB3 inhibitor is intravitreal administration.

In some embodiments, treating AMD (e.g., wAMD) according to a method provided herein comprises reducing one or more symptoms of AMD (e.g., wAMD) in the subject compared to a control subject or compared to the subject prior to treatment with the HIF1-α Pathway Inhibitor and the PFKFB3 inhibitor. In some embodiments, the one or more reduced symptoms of AMD is selected from: choroidal neovascularization, and ocular swelling and/or hemorrhage. In some embodiments, the one or more reduced symptoms of AMD is ocular swelling, hemorrhage, and or reduced central vision. In some embodiments, the one or more reduced symptoms of AMD is selected from: retinal inflammation, acellular capillary formation, choroidal neovascularization, ocular endothelial cell death, ocular vascular permeability, ocular ischemia-reperfusion injury, ocular leakage area, and occludin disruption. In some embodiments, the one or more symptoms of AMD are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the provided compositions and methods prevent or lessen the incidence, prevalence, or severity of damaged and/or leaky blood vessels in the eye.

Treatment and/or prevention of age-related macular degeneration can be measured by a variety of means. In some embodiments, treatment or prevention comprises treating one or more of apoptosis, inflammation, acellular capillary formation, neovascularization, retinal endothelial cell death, retinal vascular permeability, ischemic reperfusion injury, and occludin disruption in age-related macular degeneration in a subject in need thereof.

In some embodiments, the subject treated according to a method provided herein demonstrates stable or improved vision with 3-line or greater vision improvement on the ETDRS chart.

In some embodiments, treating AMD (e.g., wAMD) according to a method provided herein comprises increasing one or more vision parameters in the subject compared to the vision parameters in a control subject or compared to the vision parameters of the subject prior to treatment with the HIF1-α Pathway Inhibitor and the PFKFB3 inhibitor. In some embodiments, the one or more increased vision parameters are selected from hemorrhage, swelling, scar tissue, and loss of central vision

The disclosure provides methods of treating AMD (e.g., wAMD) with an anti-HIF1-alpha and/or anti-PFKFB3 antibody or antigen-binding fragment thereof. Efficacy of such treatment may be characterized, evaluated, measured, and/or monitored based on several parameters.

In some embodiments, the provided methods result in decreased apoptosis and/or endothelial cell death within the eye. Cell death may be monitored according to known methods. Illustrative methods for detecting cell death include but are not limited to, nuclear staining techniques such as propidium iodide, Hoechst-33342, 4′, 6-diamidino-2-phenylindole (DAPI), and Acridine orange-Ethidium bromide staining. Nonnuclear staining techniques include but are not limited to, Annexin-V staining.

In some embodiments, the provided methods prevent ischemia-reperfusion (IR) injury. Methods for detecting IR injury are known in the art and include fluorescein analysis, fluorescent zinc 2,2′-dipicolylamine coordination complex PSVue.RTM.794, 99mTc glucarate, and electroretinography.

In some embodiments, the provided methods reduce levels of inflammatory cytokines, such as TNFα, IL-1β, IL-6, or MCP1. Cytokine levels may be monitored via enzyme-linked immunosorbant assay (ELISA), Luminex, Cytokine Bead Array, Proteo Plex, FAST Quant, and the like.

In some embodiments, the provided methods reduce ocular vascular permeability, choroidal neovascularization, or other symptoms of ocular health. In some embodiments, the provided methods may prevent typical symptoms of age-related macular degeneration in the retinal vasculature or may prevent further deterioration. Vascular permeability and other measures of retinal vascular health may be measured by, e.g., fluorescein angiography.

In some embodiments, the provided methods improve one or more vision parameters or prevent the decline of one or more vision parameters. Vision parameters include: blurred vision, visual distortion, and speckles in the visual field. In some embodiments, subjects receiving treatment according to the provided methods may experience one or more of the following effects: improved vision, reduced vision loss, improved clarity and reduced speckles in the visual field. Many of these parameters may be monitored through routine eye examination.

In some embodiments, the provided methods reduce, ameliorate, lessen the severity of, or reverse one or more symptoms of age-related macular degeneration. In some embodiments, methods of treating age-related macular degeneration with anti-HIF1-alpha and/or anti-PFKFB3 antibodies or antigen-binding fragments thereof may reduce, ameliorate, lessen the severity of, or reverse one or more of the following symptoms: microaneurysms, hemorrhages, intraocular microvascular abnormalities, the formation of new blood vessels (neovascularization) impaired central vision, and intraocular (e.g., choroidal) microvascular abnormalities.

In some embodiments, the provided methods of treating age-related macular degeneration affect one or more parameters of the choroidal vasculature, including, but not limited to: permeability, NFkB levels, inflammatory marker levels, apoptosis incidence, sphingolipid metabolism, and reendothelialization. In some embodiments, a method of treating age-related macular degeneration disclosed herein decreases choroidal vascular permeability. Choroidal vascular permeability may be monitored via fluorescence, tracer dyes, optical exam, and the like. In some embodiments, a method of treating age-related macular degeneration disclosed herein decreases NFkB and/or other inflammatory marker levels in the choroidal vasculature. As discussed above, inflammatory cytokine levels may be measured through conventional means (e.g., ELISA). In some embodiments, a method of treating age-related macular degeneration disclosed herein decreases the incidence of apoptosis in the choroidal vasculature. As disclosed above, apoptosis may be measured using conventional means, e.g., nuclear and nonnuclear staining techniques.

In additional embodiments, the provided methods include further administering an anti-VEGF therapeutic agent to the subject. In some embodiments, the anti-VEGF therapeutic agent is bevacizumab, ranibizumab, or aflibercept. In one embodiment, the anti-VEGF therapeutic agent is bevacizumab. In one embodiment, the anti-VEGF therapeutic agent is ranibizumab. In one embodiment, the anti-VEGF therapeutic agent is aflibercept.

In some embodiments, the subject receiving a treatment provided herein has received a prior treatment for AMD (e.g., wAMD). In some embodiments, the prior treatment is a therapeutic procedure selected from a vitrectomy and laser surgery, or a therapeutic agent selected from a steroid and an anti-vascular endothelial growth factor (VEGF) therapy. In a particular embodiment, the prior treatment is an anti-vascular endothelial growth factor (VEGF) therapy. In some embodiments, the prior anti-VEGF therapy included bevacizumab, ranibizumab, or aflibercept. In one embodiment, the prior anti-VEGF therapy included bevacizumab. In one embodiment, the prior anti-VEGF therapy included ranibizumab. In one embodiment, the prior anti-VEGF therapy included aflibercept. In some embodiments, the subject failed to respond to the prior treatment for AMD. In such embodiments, a “failure to respond” indicates that the previous treatment failed to ameliorate and/or improve one or more symptoms of age-related macular degeneration. In such embodiments, a “failure to respond” indicates that the previous treatment failed to ameliorate and/or improve one or more symptoms of age-related macular degeneration. In some embodiments, the previous therapy may have failed to produce an improvement in vision or in retinal vascular health. In some embodiments, the previous therapy may have shown some results, but may not have achieved the desired performance or may have stopped showing efficacy after some period of time. In some embodiments, the subject may have partially responded to a previous age-related macular degeneration treatment: i.e., one or more symptoms of age-related macular degeneration were not sufficiently ameliorated and/or the effects of the previous treatment were not sufficiently durable.

Existing treatments for age-related macular degeneration include anti-VEGF therapy.

### Choroidal Neovascular Membranes

In some embodiments, the disclosure provides methods and compositions for treating Choroidal neovascular membranes (CNVM).

Choroidal neovascular membranes are associated with new, damaging blood vessels that grow beneath the retina in an area called the choroid. The blood vessels break through the barrier between the choroid and the retina. When they leak or bleed in the retina they cause vision loss. CNVM is often associated with wet age-related macular degeneration and is also found in patients with disorders that include histoplasmosis, eye injury and myopic macular degeneration.

In some embodiments, the disclosure provides compositions and methods for treating choroidal neovascular membranes (CNVM). In one embodiment, that disclosure provides a method of treating CNVM in a subject in need thereof comprising: (a) administering an effective amount of a HIF1-α Pathway Inhibitor and an PFKFB3 inhibitor to the subject; (b) administering an effective amount of a HIF1-α Pathway Inhibitor to the subject, wherein the subject has previously been administered a PFKFB3 Inhibitor; or (c) administering an effective amount of a PFKFB3 Inhibitor to the subject, wherein the subject has previously been administered a HIF1-α Pathway Inhibitor; wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having CNVM. In some embodiments the subject is at risk of having CNVM. In some embodiments, a method provided herein (e.g., and of (a)-(c) above), is performed as a prophylactic treatment for CNVM.

In some embodiments, the provided methods and compositions prevent choroidal neovascular membranes in a subject at risk for developing CNVM, e.g., a subject having one or more risk factors associated with development of choroidal neovascular membranes. In some embodiments, the subject has AMD. In some embodiments, the subject has wAMD. In some embodiments the subject has ocular histoplasmosis or pathologic myopia.

In some embodiments, the HIF1-α Pathway Inhibitor and the PFKFB3 inhibitor are administered before the onset of one or more symptoms of CNVM. In some embodiments, the provided compositions and methods may reduce the incidence, severity, or level of choroidal neovascularization, ocular bleeding, hemorrhage or loss of vision.

In some embodiments, treating CNVM according to a method provided herein comprises delaying the onset of one or more symptoms of CNVM.

In some embodiments, the HIF1-α Pathway Inhibitor and the PFKFB3 inhibitor are administered after the onset of one or more symptoms of CNVM.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1.3, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968, nanobody AG-1-5 or nanobody AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating CNVM is performed by co-administering the HIF1-α Pathway Inhibitor and the PFKFB3 inhibitor to the subject.

In some embodiments, the administration of the HIF1-α Pathway Inhibitor or the PFKFB3 inhibitor is an ocular administration. In some embodiments, the administration of the HIF1-α Pathway Inhibitor and the PFKFB3 inhibitor is an ocular administration. In some embodiments, the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

In some embodiments, the administration of the HIF1-α Pathway Inhibitor or the PFKFB3 inhibitor is an intravitreal administration. In some embodiments, the administration of the HIF1-α Pathway Inhibitor and the PFKFB3 inhibitor is intravitreal administration.

In some embodiments, treating CNVM according to a method provided herein comprises reducing one or more symptoms of CNVM in the subject compared to a control subject or compared to the subject prior to treatment with the HIF1-α Pathway Inhibitor and the PFKFB3 inhibitor. In some embodiments, the one or more reduced symptoms of CNVM is selected from: choroidal neovascularization, and ocular swelling and/or hemorrhage. In some embodiments, the one or more reduced symptoms of CNVM is ocular swelling, hemorrhage, and or reduced central vision. In some embodiments, the one or more reduced symptoms of CNVM is selected from: retinal inflammation, acellular capillary formation, choroidal neovascularization, ocular endothelial cell death, ocular vascular permeability, ocular ischemia-reperfusion injury, ocular leakage area, and occludin disruption. In some embodiments, the one or more symptoms of CNVM are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the provided compositions and methods prevent or lessen the incidence, prevalence, or severity of damaged and/or leaky blood vessels in the eye.

Treatment and/or prevention of choroidal neovascular membranes can be measured by a variety of means. In some embodiments, treatment or prevention comprises treating one or more of apoptosis, inflammation, acellular capillary formation, neovascularization, retinal endothelial cell death, retinal vascular permeability, ischemic reperfusion injury, and occludin disruption in choroidal neovascular membranes in a subject in need thereof.

In some embodiments, the subject treated according to a method provided herein demonstrates stable or improved vision with 3-line or greater vision improvement on the ETDRS chart.

In some embodiments, treating CNVM according to a method provided herein comprises increasing one or more vision parameters in the subject compared to the vision parameters in a control subject or compared to the vision parameters of the subject prior to treatment with the HIF1-α Pathway Inhibitor and the PFKFB3 inhibitor. In some embodiments, the one or more increased vision parameters are selected from hemorrhage, swelling, scar tissue, and loss of central vision

The disclosure provides methods of treating CNVM with an anti-HIF1-alpha and/or anti-PFKFB3 antibody or antigen-binding fragment thereof. Efficacy of such treatment may be characterized, evaluated, measured, and/or monitored based on several parameters.

CNVM can routinely be diagnosed and monitored using diagnostic tests such as Optical Coherence Tomography (OCT) or fluorescein angiography.

In some embodiments, the provided methods result in decreased apoptosis and/or endothelial cell death within the eye. Cell death may be monitored according to known methods. Illustrative methods for detecting cell death include but are not limited to, nuclear staining techniques such as propidium iodide, Hoechst-33342, 4′, 6-diamidino-2-phenylindole (DAPI), and Acridine orange-Ethidium bromide staining. Nonnuclear staining techniques include but are not limited to, Annexin-V staining.

In some embodiments, the provided methods prevent ischemia-reperfusion (IR) injury. Methods for detecting IR injury are known in the art and include fluorescein analysis, fluorescent zinc 2,2′-dipicolylamine coordination complex PSVue.RTM.794, 99mTc glucarate, and electroretinography.

In some embodiments, the provided methods reduce levels of inflammatory cytokines, such as TNFα, IL-1β, IL-6, or MCP1. Cytokine levels may be monitored via enzyme-linked immunosorbant assay (ELISA), Luminex, Cytokine Bead Array, Proteo Plex, FAST Quant, and the like.

In some embodiments, the provided methods reduce ocular vascular permeability, choroidal neovascularization, or other symptoms of ocular health. In some embodiments, the provided methods may prevent typical symptoms of choroidal neovascular membranes in the retinal vasculature or may prevent further deterioration. Vascular permeability and other measures of retinal vascular health may be measured by, e.g., fluorescein angiography.

In some embodiments, the provided methods improve one or more vision parameters or prevent the decline of one or more vision parameters. Vision parameters include: blurred vision, visual distortion, and speckles in the visual field. In some embodiments, subjects receiving treatment according to the provided methods may experience one or more of the following effects: improved vision, reduced vision loss, improved clarity and reduced speckles in the visual field. Many of these parameters may be monitored through routine eye examination.

In some embodiments, the provided methods reduce, ameliorate, lessen the severity of, or reverse one or more symptoms of choroidal neovascular membranes. In some embodiments, methods of treating choroidal neovascular membranes with anti-HIF1-alpha and/or anti-PFKFB3 antibodies or antigen-binding fragments thereof may reduce, ameliorate, lessen the severity of, or reverse one or more of the following symptoms: microaneurysms, hemorrhages, intraocular microvascular abnormalities, the formation of new blood vessels (neovascularization) impaired central vision, and intraocular (e.g., choroidal) microvascular abnormalities.

In some embodiments, the provided methods of treating choroidal neovascular membranes affect one or more parameters of the choroidal vasculature, including, but not limited to: permeability, NFkB levels, inflammatory marker levels, apoptosis incidence, sphingolipid metabolism, and reendothelialization. In some embodiments, a method of treating choroidal neovascular membranes disclosed herein decreases choroidal vascular permeability. Choroidal vascular permeability may be monitored via fluorescence, tracer dyes, optical exam, and the like. In some embodiments, a method of treating choroidal neovascular membranes disclosed herein decreases NFkB and/or other inflammatory marker levels in the choroidal vasculature. As discussed above, inflammatory cytokine levels may be measured through conventional means (e.g., ELISA). In some embodiments, a method of treating choroidal neovascular membranes disclosed herein decreases the incidence of apoptosis in the choroidal vasculature. As disclosed above, apoptosis may be measured using conventional means, e.g., nuclear and nonnuclear staining techniques.

In additional embodiments, the provided methods include further administering an anti-VEGF therapeutic agent to the subject. In some embodiments, the anti-VEGF therapeutic agent is bevacizumab, ranibizumab, or aflibercept. In one embodiment, the anti-VEGF therapeutic agent is bevacizumab. In one embodiment, the anti-VEGF therapeutic agent is ranibizumab. In one embodiment, the anti-VEGF therapeutic agent is aflibercept.

In some embodiments, the subject receiving a treatment provided herein has received a prior treatment for CNVM. In some embodiments, the prior treatment is a therapeutic procedure selected from a vitrectomy and laser surgery, or a therapeutic agent selected from a steroid and an anti-vascular endothelial growth factor (VEGF) therapy. In a particular embodiment, the prior treatment is an anti-vascular endothelial growth factor (VEGF) therapy. In some embodiments, the prior anti-VEGF therapy included bevacizumab, ranibizumab, or aflibercept. In one embodiment, the prior anti-VEGF therapy included bevacizumab. In one embodiment, the prior anti-VEGF therapy included ranibizumab. In one embodiment, the prior anti-VEGF therapy included aflibercept. In some embodiments, the subject failed to respond to the prior treatment for CNVM. In such embodiments, a “failure to respond” indicates that the previous treatment failed to ameliorate and/or improve one or more symptoms of choroidal neovascular membranes. In such embodiments, a “failure to respond” indicates that the previous treatment failed to ameliorate and/or improve one or more symptoms of choroidal neovascular membranes. In some embodiments, the previous therapy may have failed to produce an improvement in vision or in retinal vascular health. In some embodiments, the previous therapy may have shown some results, but may not have achieved the desired performance or may have stopped showing efficacy after some period of time. In some embodiments, the subject may have partially responded to a previous choroidal neovascular membranes treatment: i.e., one or more symptoms of choroidal neovascular membranes were not sufficiently ameliorated and/or the effects of the previous treatment were not sufficiently durable.

Existing treatments for choroidal neovascular membranes include laser photocoagulation, photodynamic therapy, and anti-VEGF therapy (e.g., bevacizumab (AVASTIN®), ranibizumab (e.g., LUCENTIS®), and pegaptanib (MACUGEN®).

The disclosure of each of U.S. Appl. No. 63/189,204, U.S. Appl. No. 63/189,205, U.S. Appl. No. 63/189,206, U.S. Appl. No. 63/189,207, each filed May 16, 2021, is herein incorporated by reference in its entirety.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method of treating an ocular neovascular disease or condition in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

2. The method of claim 1, wherein the subject is administered an effective amount of the HIF1-α Pathway Inhibitor and the PFKFB3 inhibitor.

3. The method of claim 1, wherein the subject is administered an effective amount of the HIF1-α Pathway Inhibitor and wherein the subject has previously been administered the PFKFB3 Inhibitor.

4. The method of claim 1, wherein the subject is administered an effective amount of the PFKFB3 Inhibitor and wherein the subject has previously been administered the HIF1-α Pathway Inhibitor.

5. The method of any one of claims 1-4, wherein the subject has or is at risk of having the ocular neovascular disease or condition.

6. The method of any one of claims 1-4, wherein the subject has or has been diagnosed as having the ocular neovascular disease or condition.

7. The method of any one of claims 1-5, wherein the method of any one of 1(a)-1(c) is administered as a prophylactic treatment for the ocular neovascular disease or condition.

8. The method of any one of claims 1-7, wherein the ocular neovascular disease or condition is diabetic retinopathy (DR).

9. The method of any one of claims 1-8, wherein the ocular neovascular disease or condition is diabetic macular edema (DME).

10. The method of any one of claims 1-7, wherein the ocular neovascular disease or condition is age-related macular degeneration (AMD), such as wet AMD (wAMD).

11. The method of any one of claims 1-7, wherein the ocular neovascular disease or condition is choroidal neovascular membranes.

12. The method of any one of claims 1-11 wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor.

13. The method of any one of claims 1-12 wherein the administered HIF1-α Pathway Inhibitor is silibinin, PX-478 or YC-1.3, or a salt thereof.

14. The method of any one of claims 1-13 wherein the administered HIF1-α Pathway Inhibitor is ganetespib (ST-9090), phenethyl isothiocyanate, or BAY-87-2243, or a salt thereof.

15. The method of any one of claims 1-14 wherein the administered HIF1-α Pathway Inhibitor is a HIF1-α Inhibitor.

16. The method of claim 15 wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

17. The method of claim 15 or 16, wherein the administered HIF1-α Inhibitor is antisense oligonucleotide EZN-2968 or nanobody AG-1, AG-2, AG-3, AG-4, AG-5, VHH212, or AHPC.

18. The method of any one of claims 1-17, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

19. The method of any one of claims 1-18, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

20. The method of any one of claims 1-19, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

21. The method of any one of claims 1-20, wherein the HIF1-α Pathway Inhibitor and the PFKFB3 inhibitor are co-administered to the subject.

22. The method of any one of claims 1-21, wherein the administration of the HIF1-α Pathway Inhibitor or the PFKFB3 inhibitor is an ocular administration.

23. The method of any one of claims 1-22, wherein the administration of the HIF1-α Pathway Inhibitor and the PFKFB3 inhibitor is an ocular administration.

24. The method of claim 22 or 23, wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

25. The method of any one of claims 22-24, wherein the administration of the HIF1-α Pathway Inhibitor or the PFKFB3 inhibitor is intravitreal administration.

26. The method of any one of claims 22-25, wherein the administration of the HIF1-α Pathway Inhibitor and the PFKFB3 inhibitor is intravitreal administration.

27. The method of any one of claims 1-26, wherein the HIF1-α Pathway Inhibitor and the PFKFB3 inhibitor are administered before the onset of one or more symptoms of the ocular neovascular disease or condition.

28. The method of any one of claims 1-26, wherein the HIF1-α Pathway Inhibitor and the PFKFB3 inhibitor are administered after the onset of one or more symptoms of the ocular neovascular disease or condition.

29. The method of any one of claims 1-26, wherein the HIF1-α Pathway Inhibitor and the PFKFB3 inhibitor are administered during a non-proliferative stage of the ocular neovascular disease or condition.

30. The method of any one of claims 1-26, wherein the HIF1-α Pathway Inhibitor and the PFKFB3 inhibitor are administered during a proliferative stage of the ocular neovascular disease or condition.

31. The method of any one of claims 1-27, wherein treating the ocular neovascular disease or condition comprises delaying the onset of the ocular neovascular disease or condition.

32. The method of any one of claims 1-31, wherein one or more symptoms of the ocular neovascular disease or condition is reduced in the subject compared to a control subject or compared to the subject prior to treatment with the HIF1-α Pathway Inhibitor and the PFKFB3 inhibitor.

33. The method of claim 32, wherein the one or more symptoms of the ocular neovascular disease or condition is selected from: retinal inflammation, acellular capillary formation, retinal neovascularization, retinal endothelial cell death, retinal vascular permeability, retinal ischemia-reperfusion injury, retinal leakage area, choroidal inflammation, choroidal neovascularization, choroidal endothelial cell death, choroidal vascular permeability, choroidal ischemia-reperfusion injury, choroidal leakage area, and occludin disruption and occludin disruption.

34. The method of claim 32 or 33, wherein the one or more symptoms of the ocular neovascular disease or condition are reduced by at least 10%, at least 20%, at least 30%, at least 40%, or at least 50% compared to the control subject or compared to the subject prior to treatment with the HIF1-α Pathway Inhibitor and the PFKFB3 inhibitor.

35. The method of any one of claims 1-34, wherein one or more vision parameters are increased in the subject compared to the vision parameters in a control subject or compared to the vision parameters of the subject prior to treatment with the HIF1-α Pathway Inhibitor and the PFKFB3 inhibitor.

36. The method of claim 35, wherein the one or more vision parameters are selected from peripheral vision; night vision; low light vision, color vision; distance vision; close-range vision; vision field clarity, ability to read, absence of flashing lights or spots in the vision filed, non-fluctuating vision, pain, and eye appearance.

37. The method of any one of claims 1-36, which further comprises administering an anti-VEGF therapeutic agent to the subject.

38. The method of claim 37, wherein the anti-VEGF therapeutic agent is bevacizumab, ranibizumab, or aflibercept.

39. The method of any one of claims 1-38, wherein the subject has received a prior treatment for the ocular neovascular disease or condition.

40. The method of claim 39, wherein the subject failed to respond to the prior treatment for the ocular neovascular disease or condition.

41. The method of claim 39 or 40, wherein the prior treatment is a therapeutic procedure selected from a vitrectomy and laser surgery, or a therapeutic agent selected from a steroid and an anti-vascular endothelial growth factor (VEGF) therapy.

42. The method of claim 41, wherein the prior treatment is an anti-vascular endothelial growth factor (VEGF) therapy.

43. The method of claim 42, wherein the anti-VEGF therapy is the administration of bevacizumab, ranibizumab, or aflibercept.

44. A method of treating diabetic retinopathy (DR) in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

45. The method of claim 44, wherein the subject is administered an effective amount of the HIF1-α Pathway Inhibitor and the PFKFB3 inhibitor.

46. The method of claim 44, wherein the subject is administered an effective amount of the HIF1-α Pathway Inhibitor and wherein the subject has previously been administered the PFKFB3 Inhibitor.

47. The method of claim 44, wherein the subject is administered an effective amount of the PFKFB3 Inhibitor and wherein the subject has previously been administered the HIF1-α Pathway Inhibitor.

48. The method of any one of claims 44-47, wherein the subject has or is at risk of having DR.

49. The method of any one of claims 44-48, wherein the subject has or has been diagnosed as having DR.

50. The method of any one of claims 44-48, wherein the method of any one of 44(a)-44(c) is administered as a prophylactic treatment for DR.

51. The method of any one of claims 44-50 wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

52. The method of any one of claims 44-51 wherein the administered HIF1-α Pathway Inhibitor is silibinin, PX-478 or YC-1.3, or a salt thereof.

53. The method of any one of claims 44-52 wherein the administered HIF1-α Pathway Inhibitor is ganetespib (ST-9090), phenethyl isothiocyanate, or BAY-87-2243, or a salt thereof.

54. The method of any one of claims 44-53 wherein the administered HIF1-α Pathway Inhibitor is a HIF1-α Inhibitor.

55. The method of claim 54, wherein the HIF1-α Inhibitor is HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

56. The method of claim 54 or 55, wherein the administered HIF1-α Inhibitor is antisense oligonucleotide EZN-2968 or nanobody AG-1, AG-2, AG-3, AG-4, AG-5, VHH212, or AHPC.

57. The method of any one of claims 44-56, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

58. The method of any one of claims 44-57, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

59. The method of any one of claims 44-57, wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

60. The method of any one of claims 44-59, wherein the HIF1-α Pathway Inhibitor and the PFKFB3 inhibitor are co-administered to the subject.

61. The method of any one of claims 44-60, wherein the administration of the HIF1-α Pathway Inhibitor or the PFKFB3 inhibitor is an ocular administration.

62. The method of any one of claims 44-61, wherein the administration of the HIF1-α Pathway Inhibitor and the PFKFB3 inhibitor is an ocular administration.

63. The method of claim 61 or 62, wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

64. The method of any one of claims 61-63, wherein the administration of the HIF1-α Pathway Inhibitor or the PFKFB3 inhibitor is intravitreal administration.

65. The method of any one of claims 61-64, wherein the administration of the HIF1-α Pathway Inhibitor and the PFKFB3 inhibitor is intravitreal administration.

66. The method of any one of claims 44-65, wherein the HIF1-α Pathway Inhibitor and the PFKFB3 inhibitor are administered before the onset of one or more symptoms of DR.

67. The method of any one of claims 44-65, wherein the HIF1-α Pathway Inhibitor and the PFKFB3 inhibitor are administered after the onset of one or more symptoms of DR.

68. The method of any one of claims 44-65, wherein the HIF1-α Pathway Inhibitor and the PFKFB3 inhibitor are administered during a non-proliferative stage of DR.

69. The method of any one of claims 44-65 wherein the HIF1-α Pathway Inhibitor and the PFKFB3 inhibitor are administered during a proliferative stage of DR.

70. The method of any one of claims 44-69, wherein treating DR comprises delaying the onset of DR.

71. The method of any one of claims 44-70 wherein one or more symptoms of DR is reduced in the subject compared to a control subject or compared to the subject prior to treatment with the HIF1-α Pathway Inhibitor and the PFKFB3 inhibitor.

72. The method of claim 71, wherein the one or more symptoms of DR is selected from: retinal inflammation, acellular capillary formation, retinal neovascularization, retinal endothelial cell death, retinal vascular permeability, retinal ischemia-reperfusion injury, retinal leakage area, and occludin disruption.

73. The method of claim 71 or 72, wherein the one or more symptoms of DR are reduced by at least 10%, at least 20%, at least 30%, at least 40%, or at least 50% compared to the control subject or compared to the subject prior to treatment with the HIF1-α Pathway Inhibitor and the PFKFB3 inhibitor.

74. The method of any one of claims 44-73, wherein one or more vision parameters are increased in the subject compared to the vision parameters a in a control subject or compared to the vision parameters of the subject prior to treatment with the HIF1-α Pathway Inhibitor and the PFKFB3 inhibitor.

75. The method of claim 74, wherein the one or more vision parameters are selected from peripheral vision; night vision; color vision; distance vision; close-range vision; and vision clarity.

76. The method of any one of claims 44-75, which further comprises administering an anti-VEGF therapeutic agent to the subject.

77. The method of claim 76, wherein the anti-VEGF therapeutic agent is bevacizumab, ranibizumab, or aflibercept.

78. The method of any one of claims 44-77, wherein the subject has received a prior treatment for DR.

79. The method of claim 78, wherein the subject failed to respond to the prior treatment for DR.

80. The method of claim 78 or 79, wherein the prior treatment is a therapeutic procedure selected from a vitrectomy and laser surgery, or a therapeutic agent selected from a steroid and an anti-vascular endothelial growth factor (VEGF) therapy.

81. The method of claim 79 or 80, wherein the prior treatment is an anti-vascular endothelial growth factor (VEGF) therapy.

82. The method of claim 81, wherein the anti-VEGF therapy is the administration of bevacizumab, ranibizumab, or aflibercept.

83. A method of treating Diabetic macular edema (DME) in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

84. The method of claim 83, wherein the subject is administered an effective amount of the HIF1-α Pathway Inhibitor and the PFKFB3 inhibitor.

85. The method of claim 83, wherein the subject is administered an effective amount of the HIF1-α Pathway Inhibitor and wherein the subject has previously been administered the PFKFB3 Inhibitor.

86. The method of claim 83, wherein the subject is administered an effective amount of the PFKFB3 Inhibitor and wherein the subject has previously been administered the HIF1-α Pathway Inhibitor.

87. The method of any one of claims 83-86, wherein the subject has or is at risk of having DME.

88. The method of any one of claims 83-87, wherein the subject has or has been diagnosed as having DME.

89. The method of any one of claims 83-87, wherein the method of any one of 83(a)-83(c) is administered as a prophylactic treatment for DME.

90. The method of any one of claims 83-89 wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

91. The method of any one of claims 83-90 wherein the administered HIF1-α Pathway Inhibitor is silibinin, PX-478 or YC-1.3, or a salt thereof.

92. The method of any one of claims 83-91 wherein the administered HIF1-α Pathway Inhibitor is ganetespib (ST-9090), phenethyl isothiocyanate, or BAY-87-2243, or a salt thereof.

93. The method of any one of claims 83-92 wherein the administered HIF1-α Pathway Inhibitor is a HIF1-α Inhibitor.

94. The method of claim 93, wherein the HIF1-α Inhibitor is HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

95. The method of claim 93 or 94, wherein the administered HIF1-α Inhibitor is antisense oligonucleotide EZN-2968 or nanobody AG-1, AG-2, AG-3, AG-4, AG-5, VHH212, or AHPC.

96. The method of any one of claims 83-95, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

97. The method of any one of claims 83-96, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

98. The method of any one of claims 83-96, wherein the administered PFKFB3 Inhibitor is (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

99. The method of any one of claims 83-98, wherein the HIF1-α Pathway Inhibitor and the PFKFB3 inhibitor are co-administered to the subject.

100. The method of any one of claims 83-99, wherein the administration of the HIF1-α Pathway Inhibitor or the PFKFB3 inhibitor is an ocular administration.

101. The method of any one of claims 83-100, wherein the administration of the HIF1-α Pathway Inhibitor and the PFKFB3 inhibitor is an ocular administration.

102. The method of claim 100 or 101, wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

103. The method of any one of claims 100-102, wherein the administration of the HIF1-α Pathway Inhibitor or the PFKFB3 inhibitor is intravitreal administration.

104. The method of any one of claims 100-103, wherein the administration of the HIF1-α Pathway Inhibitor and the PFKFB3 inhibitor is intravitreal administration.

105. The method of any one of claims 83-104, wherein the HIF1-α Pathway Inhibitor and the PFKFB3 inhibitor are administered before the onset of one or more symptoms of DME.

106. The method of any one of claims 83-104, wherein the HIF1-α Pathway Inhibitor and the PFKFB3 inhibitor are administered after the onset of one or more symptoms of DME.

107. The method of any one of claims 83-104, wherein the HIF1-α Pathway Inhibitor and the PFKFB3 inhibitor are administered during a non-proliferative stage of DME.

108. The method of any one of claims 83-104 wherein the HIF1-α Pathway Inhibitor and the PFKFB3 inhibitor are administered during a proliferative stage of DME.

109. The method of any one of claims 83-105, wherein treating DME comprises delaying the onset of DME.

110. The method of any one of claims 83-109 wherein one or more symptoms of DME is reduced in the subject compared to a control subject or compared to the subject prior to treatment with the HIF1-α Pathway Inhibitor and the PFKFB3 inhibitor.

111. The method of claim 110, wherein the one or more symptoms of DME is selected from: distorted vision, retinal inflammation, acellular capillary formation, retinal neovascularization, retinal endothelial cell death, retinal vascular permeability, retinal ischemia-reperfusion injury, retinal leakage area, rupture of the hematorretinal barrier of the retina in a retinal vascular endothelial cell or a retinal pigment epithelial cell, retinal scarring, and occludin disruption.

112. The method of claim 110 or 111, wherein the one or more symptoms of DME are reduced by at least 10%, at least 20%, at least 30%, at least 40%, or at least 50% compared to the control subject or compared to the subject prior to treatment with the HIF1-α Pathway Inhibitor and the PFKFB3 inhibitor.

113. The method of any one of claims 83-112, wherein one or more vision parameters are increased in the subject compared to the vision parameters a in a control subject or compared to the vision parameters of the subject prior to treatment with the HIF1-α Pathway Inhibitor and the PFKFB3 inhibitor.

114. The method of claim 113, wherein the one or more vision parameters are selected from visual acuity, peripheral vision; night vision; color vision; distance vision; close-range vision; and vision clarity.

115. The method of any one of claims 83-114, wherein the subject has diabetes or diabetic retinopathy.

116. The method of any one of claims 83-115, which further comprises administering an anti-VEGF therapeutic agent to the subject.

117. The method of claim 116, wherein the anti-VEGF therapeutic agent is bevacizumab, ranibizumab, or aflibercept.

118. The method of any one of claims 83-117, wherein the subject has received a prior treatment for DME.

119. The method of claim 118, wherein the subject failed to respond to the prior treatment for DME.

120. The method of claim 118 or 119, wherein the prior treatment is a therapeutic procedure selected from a vitrectomy and laser surgery, or a therapeutic agent selected from a steroid and an anti-vascular endothelial growth factor (VEGF) therapy.

121. The method of claim 118 or 119, wherein the prior treatment is an anti-vascular endothelial growth factor (VEGF) therapy.

122. The method of claim 121, wherein the anti-VEGF therapy is the administration of bevacizumab, ranibizumab, or aflibercept.

123. A method of treating age-related macular degeneration (AMD) in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

124. The method of claim 123, wherein the subject is administered an effective amount of the HIF1-α Pathway Inhibitor and the PFKFB3 inhibitor.

125. The method of claim 123, wherein the subject is administered an effective amount of the HIF1-α Pathway Inhibitor and wherein the subject has previously been administered the PFKFB3 Inhibitor.

126. The method of claim 123, wherein the subject is administered an effective amount of the PFKFB3 Inhibitor and wherein the subject has previously been administered the HIF1-α Pathway Inhibitor.

127. The method of any one of claims 123-126, wherein the subject has or is at risk of having AMD, e.g., wet AMD (wAMD).

128. The method of any one of claims 123-126, wherein the subject has or has been diagnosed as having AMD e.g., wAMD.

129. The method of any one of claims 123-127, wherein the method of any one of 123(a)-123(c) is administered as a prophylactic treatment for AMD e.g., wAMD.

130. The method of any one of claims 123-129 wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

131. The method of any one of claims 123-130 wherein the administered HIF1-α Pathway Inhibitor is silibinin, PX-478 or YC-1.3, or a salt thereof.

132. The method of any one of claims 123-131 wherein the administered HIF1-α Pathway Inhibitor is ganetespib (ST-9090), phenethyl isothiocyanate, or BAY-87-2243, or a salt thereof.

133. The method of any one of claims 123-132 wherein the administered HIF1-α Pathway Inhibitor is a HIF1-α Inhibitor.

134. The method of claim 133, wherein the HIF1-α Inhibitor is HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

135. The method of claim 133 or 134, wherein the administered HIF1-α Inhibitor is antisense oligonucleotide EZN-2968 or nanobody AG-1, AG-2, AG-3, AG-4, AG-5, VHH212, or AHPC.

136. The method of any one of claims 123-135, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

137. The method of any one of claims 123-136, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

138. The method of any one of claims 123-136, wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

139. The method of any one of claims 123-138, wherein the HIF1-α Pathway Inhibitor and the PFKFB3 inhibitor are administered to the subject.

140. The method of any one of claims 123-139, wherein the administration of the HIF1-α Pathway Inhibitor or the PFKFB3 inhibitor is an ocular administration.

141. The method of any one of claims 123-140, wherein the administration of the HIF1-α Pathway Inhibitor and the PFKFB3 inhibitor is an ocular administration.

142. The method of claim 140 or 141, wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

143. The method of any one of claims 140-142, wherein the administration of the HIF1-α Pathway Inhibitor or the PFKFB3 inhibitor is intravitreal administration.

144. The method of any one of claims 140-143, wherein the administration of the HIF1-α Pathway Inhibitor and the PFKFB3 inhibitor is intravitreal administration.

145. The method of any one of claims 123-144, wherein the HIF1-α Pathway Inhibitor and the PFKFB3 inhibitor are administered before the onset of one or more symptoms of AMD.

146. The method of any one of claims 123-144, wherein the HIF1-α Pathway Inhibitor and the PFKFB3 inhibitor are administered after the onset of one or more symptoms of AMD.

147. The method of any one of claims 123-144, wherein the HIF1-α Pathway Inhibitor and the PFKFB3 inhibitor are administered during a non-proliferative stage of AMD.

148. The method of any one of claims 123-144 wherein the HIF1-α Pathway Inhibitor and the PFKFB3 inhibitor are administered during a proliferative stage of AMD.

149. The method of any one of claims 123-145, wherein treating AMD comprises delaying the onset of AMD.

150. The method of any one of claims 123-149 wherein one or more symptoms of AMD is reduced in the subject compared to a control subject or compared to the subject prior to treatment with the HIF1-α Pathway Inhibitor and the PFKFB3 inhibitor.

151. The method of claim 150, wherein the one or more symptoms of AMD is selected from: choroidal inflammation, choroidal neovascularization, choroidal endothelial cell death, choroidal vascular permeability, choroidal ischemia-reperfusion injury, choroidal leakage area, and occludin disruption, acellular capillary formation, vascular permeability, and occludin disruption.

152. The method of claim 150 or 151, wherein the one or more symptoms of AMD are reduced by at least 10%, at least 20%, at least 30%, at least 40%, or at least 50% compared to the control subject or compared to the subject prior to treatment with the HIF1-α Pathway Inhibitor and the PFKFB3 inhibitor.

153. The method of any one of claims 123-151, wherein one or more vision parameters are increased in the subject compared to the vision parameters a in a control subject or compared to the vision parameters of the subject prior to treatment with the HIF1-α Pathway Inhibitor and the PFKFB3 inhibitor.

154. The method of claim 153, wherein the one or more vision parameters are selected from peripheral vision; night vision; color vision; distance vision; close-range vision; and vision clarity.

155. The method of any one of claims 123-154, wherein the subject has, or is at risk of having wAMD.

156. The method of any one of claims 123-155, which further comprises administering an anti-VEGF therapeutic agent to the subject.

157. The method of claim 156, wherein the anti-VEGF therapeutic agent is bevacizumab, ranibizumab, or aflibercept.

158. The method of any one of claims 123-157, wherein the subject has received a prior treatment for AMD.

159. The method of claim 158, wherein the subject failed to respond to the prior treatment for AMD.

160. The method of claim 158 or 159, wherein the prior treatment is a therapeutic procedure selected from a vitrectomy and laser surgery, or a therapeutic agent selected from a steroid and an anti-vascular endothelial growth factor (VEGF) therapy.

161. The method of claim 158 or 159, wherein the prior treatment is an anti-vascular endothelial growth factor (VEGF) therapy.

162. The method of claim 161, wherein the anti-VEGF therapy is the administration of bevacizumab, ranibizumab, or aflibercept.

163. A method of treating choroidal neovascularization (CNVM) in a subject in need thereof comprising:
(d) administering an effective amount of a HIF1-α Pathway Inhibitor and an PFKFB3 inhibitor to the subject;
(e) administering an effective amount of a HIF1-α Pathway Inhibitor to the subject, wherein the subject has previously been administered a PFKFB3 Inhibitor; or
(f) administering an effective amount of a PFKFB3 Inhibitor to the subject, wherein the subject has previously been administered a HIF1-α Pathway Inhibitor;
Wherein the PFKFB3 inhibitor does not inhibit PI3K/AKT/mTOR pathway or HIF1-α.

164. The method of claim 163, wherein the subject is administered an effective amount of the HIF1-α Pathway Inhibitor and the PFKFB3 inhibitor.

165. The method of claim 163, wherein the subject is administered an effective amount of the HIF1-α Pathway Inhibitor and wherein the subject has previously been administered the PFKFB3 Inhibitor.

166. The method of claim 163, wherein the subject is administered an effective amount of the PFKFB3 Inhibitor and wherein the subject has previously been administered the HIF1-α Pathway Inhibitor.

167. The method of any one of claims 163-166, wherein the subject has or is at risk of having CNVM.

168. The method of any one of claims 163-167, wherein the subject has or has been diagnosed as having CNVM.

169. The method of any one of claims 163-168, wherein the method of any one of 163(a)-163(c) is administered as a prophylactic treatment for CNVM.

170. The method of any one of claims 163-169 wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

171. The method of any one of claims 163-170 wherein the administered HIF1-α Pathway Inhibitor is silibinin, PX-478 or YC-1.3, or a salt thereof.

172. The method of any one of claims 163-171 wherein the administered HIF1-α Pathway Inhibitor is ganetespib (ST-9090), phenethyl isothiocyanate, or BAY-87-2243, or a salt thereof.

173. The method of any one of claims 163-172 wherein the administered HIF1-α Pathway Inhibitor is a HIF1-α Inhibitor.

174. The method of claim 173, wherein the HIF1-α Inhibitor is HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

175. The method of claim 173 or 174, wherein the administered HIF1-α Inhibitor is antisense oligonucleotide EZN-2968 or nanobody AG-1, AG-2, AG-3, AG-4, AG-5, VHH212, or AHPC.

176. The method of any one of claims 163-175, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

177. The method of any one of claims 163-176, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

178. The method of any one of claims 163-176, wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

179. The method of any one of claims 163-178, wherein the HIF1-α Pathway Inhibitor and the PFKFB3 inhibitor are co-administered to the subject.

180. The method of any one of claims 163-179, wherein the administration of the HIF1-α Pathway Inhibitor or the PFKFB3 inhibitor is an ocular administration.

181. The method of any one of claims 163-180, wherein the administration of the HIF1-α Pathway Inhibitor and the PFKFB3 inhibitor is an ocular administration.

182. The method of claim 180 or 181, wherein the ocular administration is selected from the group consisting of topical administration, intraocular administration, subconjunctival administration, intracameral administration, injection into the anterior chamber via the temporal limbus, intrastromal administration, intracorneal administration, subretinal administration, aqueous humor injection, subtenon administration, administration to the suprachoroidal space (SCS), administration to the supraciliary space, and intravitreal administration.

183. The method of any one of claims 180-182, wherein the administration of the HIF1-α Pathway Inhibitor or the PFKFB3 inhibitor is intravitreal administration.

184. The method of any one of claims 183-183, wherein the administration of the HIF1-α Pathway Inhibitor and the PFKFB3 inhibitor is intravitreal administration.

185. The method of any one of claims 163-184, wherein the HIF1-α Pathway Inhibitor and the PFKFB3 inhibitor are administered before the onset of one or more symptoms of CNVM.

186. The method of any one of claims 163-184, wherein the HIF1-α Pathway Inhibitor and the PFKFB3 inhibitor are administered after the onset of one or more symptoms of CNVM.

187. The method of any one of claims 163-184, wherein the HIF1-α Pathway Inhibitor and the PFKFB3 inhibitor are administered during a non-proliferative stage of CNVM.

188. The method of any one of claims 163-184 wherein the HIF1-α Pathway Inhibitor and the PFKFB3 inhibitor are administered during a proliferative stage of CNVM.

189. The method of any one of claims 163-185, wherein treating CNVM comprises delaying the onset of CNVM.

190. The method of any one of claims 163-189 wherein one or more symptoms of CNVM is reduced in the subject compared to a control subject or compared to the subject prior to treatment with the HIF1-α Pathway Inhibitor and the PFKFB3 inhibitor.

191. The method of claim 190, wherein the one or more symptoms of CNVM is selected from: choroidal inflammation, choroidal neovascularization, choroidal endothelial cell death, choroidal vascular permeability, choroidal ischemia-reperfusion injury, choroidal leakage area, and occludin disruption, acellular capillary formation, vascular permeability, and occludin disruption.

192. The method of claim 190 or 191, wherein the one or more symptoms of CNVM are reduced by at least 10%, at least 20%, at least 30%, at least 40%, or at least 50% compared to the control subject or compared to the subject prior to treatment with the HIF1-α Pathway Inhibitor and the PFKFB3 inhibitor.

193. The method of any one of claims 163-192, wherein one or more vision parameters are increased in the subject compared to the vision parameters a in a control subject or compared to the vision parameters of the subject prior to treatment with the HIF1-α Pathway Inhibitor and the PFKFB3 inhibitor.

194. The method of claim 193, wherein the one or more vision parameters are selected from peripheral vision; night vision; color vision; distance vision; close-range vision; and vision clarity.

195. The method of claim 193, wherein the subject has wet wAMD, histoplasmosis, an eye injury, or myopic macular degeneration.

196. The method of any one of claims 163-195, which further comprises administering an anti-VEGF therapeutic agent to the subject.

197. The method of claim 196, wherein the anti-VEGF therapeutic agent is bevacizumab, ranibizumab, or aflibercept.

198. The method of any one of claims 163-197, wherein the subject has received a prior treatment for CNVM.

199. The method of claim 198, wherein the subject failed to respond to the prior treatment for CNVM.

200. The method of claim 198 or 199, wherein the prior treatment is a therapeutic procedure selected from a vitrectomy and laser surgery, or a therapeutic agent selected from a steroid and an anti-vascular endothelial growth factor (VEGF) therapy.

201. The method of claim 198 or 199, wherein the prior treatment is an anti-vascular endothelial growth factor (VEGF) therapy.

202. The method of claim 201, wherein the anti-VEGF therapy is the administration of bevacizumab, ranibizumab, or aflibercept.
